(19) United States
(12) Patent Application Publication
(10) Pub. No.: US 2010/0168097 A1
(43) Pub. Date: Jul. 1, 2010

(54) Non-Nucleoside Reverse Transcriptase Inhibitors

(76) Inventors: Scott E. Wolkenberg, Jenkintown, PA (US); Craig W. Lindsley, Schwenksville, PA (US); Zhijian Zhao, Wilmington, PA (US); Theresa M. Williams, Harleysville, PA (US)

Correspondence Address: MERCK, P O BOX 2000, RAHWAY, NJ, 07065-0907, US

(21) Appl. No.: 11/922,672

(22) PCT Filed: Jun. 23, 2006
(86) PCT No.: PCT/US2006/024569

§ 371 Date: Dec. 20, 2007

Related U.S. Application Data
(60) Provisional application No. 60/694,735, filed on Jun. 28, 2005.
(60) Provisional application No. 60/707,283, filed on Aug. 11, 2005.

(51) Int. Cl. A61K 31/5377; C07D 403/04; A61K 31/4178; C07D 413/14; C07D 417/14; A61K 31/4245; A61K 31/4439; A61P 31/18

(52) U.S. Cl. 514/233.8; 548/312.1; 514/397; 544/143; 548/131; 514/364; 514/338; 546/270.4

(57) ABSTRACT

Compounds of Formula I: Formula (I); are HIV reverse transcriptase inhibitors, wherein R¹, R², R³, R⁴ and R⁵ are defined herein. The compounds of Formula (I) and their pharmaceutically acceptable salts are useful in the inhibition of HIV reverse transcriptase, the prophylaxis and treatment of infection by HIV and in the prophylaxis, delay in the onset, and treatment of AIDS. The compounds and their salts can be employed as ingredients in pharmaceutical compositions, optionally in combination with other antivirals, immunomodulators, antibiotics or vaccines.

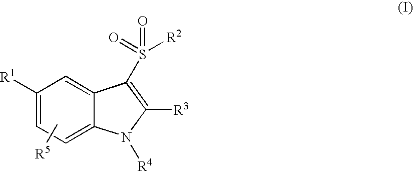

### FIELD OF THE INVENTION

The present invention is directed to certain indoles and their pharmaceutically acceptable salts and their use for the inhibition of HIV reverse transcriptase, the prophylaxis and treatment of HIV infection and HIV replication, and the prophylaxis, delay in the onset of and treatment of AIDS.

### BACKGROUND OF THE INVENTION

The retrovirus designated human immunodeficiency virus (HIV), particularly the strains known as HIV type-1 (HIV-1) and type-2 (HIV-2) viruses, have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Replication of HIV by a host cell requires integration of the viral genome into the host cell's DNA. Since HIV is a retrovirus, the HIV replication cycle requires transcription of the viral RNA genome into DNA via an enzyme know as reverse transcriptase (RT).

Reverse transcriptase has three known enzymatic functions: The enzyme acts as an RNA-dependent DNA polymerase, as a ribonuclease, and as a DNA-dependent DNA polymerase. In its role as an RNA-dependent DNA polymerase, RT transcribes a single-stranded DNA copy of the viral RNA. As a ribonuclease, RT destroys the original viral RNA and frees the DNA just produced from the original RNA. And as a DNA-dependent DNA polymerase, RT makes a second, complementary DNA strand using the first DNA strand as a template. The two strands form double-stranded DNA, which is integrated into the host cell's genome by the integrase enzyme.

It is known that compounds that inhibit enzymatic functions of HIV RT will inhibit HIV replication in infected cells. These compounds are useful in the prophylaxis or treatment of HIV infection in humans. Among the compounds approved for use in treating HIV infection and AIDS are the RT inhibitors 3′-azido-3′-deoxythymidine (An), 2′,3′-dideoxyinosine (ddI), dideoxycytidine (ddC), d4T, 3TC, nevirapine, delavirdine, efavirenz and abacavir.

While each of the foregoing drugs is effective in treating HIV infection and AIDS, there remains a need to develop additional HIV antiviral drugs including additional RT inhibitors. A particular problem is the development of mutant HIV strains that are resistant to the known inhibitors. The use of RT inhibitors to treat AIDS often leads to viruses that are less sensitive to the inhibitors. This resistance is typically the result of mutations that occur in the reverse transcriptase segment of the pol gene. The continued use of antiviral compounds to prevent HIV infection will inevitably result in the emergence of new resistant strains of HIV. Accordingly, there is a particular need for new RT inhibitors that are effective against mutant HIV strains.

The following references are of interest as background:

Williams et al., J. Med. Chem. 1993, vol. 36, pp. 1291-1294 discloses 5-chloro-3-(phenylsulfonyl)indole-2-carboxamide as a non-nucleoside inhibitor of HIV-1 reverse transcriptase.

Young et al., Bioorg. & Med. Chem. Letters 1995, vol. 5, pp. 491-496 discloses certain 2-heterocyclic indole-3-sulfones as inhibitors of HIV-1 reverse transcriptase.

GB 2,282,808 discloses certain 2-heterocyclic indole-3-sulfones as inhibitors of HIV reverse transcriptase and its resistant varieties.

U.S. Pat. No. 5,527,819 discloses certain 2-acyl substituted indole-3-sulfones as inhibitors of HIV reverse transcriptase.
WO 02/083216 A1 and WO 2004/014364 A1 each disclose certain substituted phenylindoles for the treatment of HIV.

U.S. Pat. No. 5,190,968; U.S. Pat. No. 5,204,344; U.S. Pat. No. 5,252,585; U.S. Pat. No. 5,272,145; U.S. Pat. No. 5,273,980; U.S. Pat. No. 5,290,798; U.S. Pat. No. 5,380,850; and U.S. Pat. No. 5,389,650 disclose certain indoles as inhibitors of leukotriene biosynthesis.

WO 03/024969 A1 discloses certain indazolylindole compounds as tyrosine kinase inhibitors.

WO03/099206 A2 discloses certain 2-substituted 5-oxazolyl indole compounds useful as inhibitors of IMPDH enzyme.

US 2003/0078288 A1discloses certain indole derivatives having certain substituted phenyl groups attached to the 5-position of the indole ring via O, S, S(O), S(O)₂, CH₂, CHF, CF₂, or N(C₁₋₄ alkyl). The derivatives are said to be useful for treating all indications which can be treated with natural thyroid hormones.

US 2003/0195244 A1 discloses certain indole compounds having anti-cancer activities, including certain compounds having (3,4,5-trimethoxyphenyl)sulfonyl or (3,4,5-trimethoxyphenyl)carbonyl substituted at the 3-position of the indole ring.

### SUMMARY OF THE INVENTION

The present invention is directed to certain 2-heteroarylindoles and their use in the inhibition of HIV reverse transcriptase, the prophylaxis of infection by HIV, the treatment of infection by HIV, and the prophylaxis, treatment, and delay in the onset of AIDS and/or ARC. More particularly, the present invention includes compounds of Formula I and pharmaceutically acceptable salts thereof:

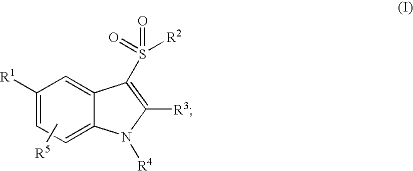

wherein:
R¹ is:
   (1) halogen,
   (2) CN,
   (3) NO₂,
   (4) C(O)R^{A},
   (5) C(O)OR^{A},
   (6) C(O)N(R^{A})R^{B},
   (7) SR^{A},
   (8) S(O)R^{A},
   (9) S(O)₂R^{A},
   (10) S(O)₂N(R^{A})R^{B},
   (11) N(R^{A})R^{B},
   (12) N(R^{A})S(O)₂R^{B},
   (13) N(R^{A})C(O)R^{B},
   (14) N(R^{A})C(O)OR^{B},
   (15) N(R^{A})S(O)₂N(R^{A})R^{B},
   (16) OC(O)N(R^{A})R^{B},
   (17) N(R^{A})C(O)N(R^{A})R^{B},
   (18) C₁₋₆ alkyl,
   (19) C₁₋₆ haloalkyl,
   (20) C₂₋₆ alkenyl,
   (21) C₂₋₆ alkynyl,
   (22) OH,
   (23) O—C₁₋₆ alkyl,
   (24) O—C₁₋₆ haloalkyl,
   (25) C₁₋₆ alkyl substituted with OH, O—C₁₋₆ alkyl, O—C₁₋₆ haloalkyl, CN, NO_{2,} N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)²R^{B}, N(R^{A})S(O)₂N(R^{A})R^{B}, OC(O)N(R^{A})R^{B}, or N(R^{A})C(O)N(R^{A})R^{B},
   (26) CycA,
   (27) AryA,
   (28) HetA,
   (29) HetR,
   (30) C₁₋₆ alkyl substituted with CycA, AryA, HetA, or HetR,
   (31) J-CycA,
   (32) J-AryA,
   (33) J-HetA, or
   (34) J-HetR;
J is:
   (1) O,
   (2) S,
   (3) S(O),
   (4) S(O)₂,
   (5) O—C₁₋₆ alkylene,
   (6) S—C₁₋₆ alkylene,
   (7) S(O)—C₁₋₆ alkylene,
   (8) S(O)₂—C₁₋₆ alkylene,
   (9) N(R^{A}),
   (10) N(R^{A})—C₁₋₆ alkylene,
   (11) C(O),
   (12) C(O)—C₁₋₆ alkylene,
   (13) C(O)—C₁₋₆ alkylene-O,
   (14) C(O)N(R^{A}),
   (15) C(O)N(R^{A})—C₁₋₆ alkylene,
   (16) C(O)N(R^{A})—C₁₋₆ alkylene-C(O)O, or
   (17) C(O)N(R^{A})S(O)₂;
CycA is C₃₋₈ cycloalkyl which is optionally substituted with a total of from 1 to 6 substituents, wherein:
   (i) from zero to 6 substituents are each independently:
      (1) halogen,
      (2) CN
      (3) C₁₋₆ alkyl,
      (4) OH,
      (5) O—C₁₋₆ alkyl,
      (6) C₁₋₆ haloalkyl, or
      (7) O—C₁₋₆ haloalkyl, and
   (ii) from zero to 2 substituents are each independently:
      (1) CycE,
      (2) AryE,
      (3) O-AryE,
      (4) HetE,
      (5) HetF, or
      (6) C₁₋₆ alkyl substituted with CycE, AryE, O-AryE, HetE, O-HetE, or HetF;
AryA is aryl which is optionally substituted with a total of from 1 to 6 substituents, wherein:
   (i) from zero to 6 substituents are each independently:
      (1) C₁₋₆ alkyl,
      (2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
      (3) O—C₁₋₆ alkyl,
      (4) C₁₋₆ haloalkyl,
      (5) O—C₁₋₆ haloalkyl,
      (6) OH,
      (7) halogen,
      (8) CN,
      (9) NO₂,
      (10) N(R^{A})R^{B},
      (11) C(O)N(R^{A})R^{B},
      (12) C(O)R^{A},
      (13) C(O)—C₁₋₆ haloalkyl,
      (14) C(O)OR^{A},
      (15) OC(O)N(R^{A})R^{B},
      (16) SR^{A},
      (17) S(O)R^{A},
      (18) S(O)₂R^{A},
      (19) S(O)₂N(R^{A})R^{B},
      (20) N(R^{A})S(O)₂R^{B},
      (21) N(R^{A})S(O)₂N(R^{A})R^{B},
      (22) N(R^{A})C(O)R^{B},
      (23) N(R^{A})C(O)N(R^{A})R^{B},
      (24) N(R^{A})C(O)—C(O)N(R^{A})R^{B}, or
      (25) N(R^{A})CO₂R^{B}, and
   (ii) from zero to 2 substituents are each independently:
      (1) CycE,
      (2) AryE,
      (3) O-AryE,
      (4) HetE,
      (5) HetF, or
      (6) C₁₋₆ alkyl substituted with CycE, AryE, O-AryE, HetE, O-HetE, or HetF;
HetA is heteroaryl which is optionally substituted with a total of from 1 to 6 substituents, wherein:
   (i) from zero to 6 substituents are each independently:
      (1) C₁₋₆ alkyl,
      (2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
      (3) C₁₋₆ alkyl substituted with from 2 to 4 OH,
      (4) O—C₁₋₆ alkyl
      (5) C₁₋₆ haloalkyl,
      (6) O—C₁₋₆ haloalkyl,
      (7) OH,
      (8) oxo,
      (9) halogen,
      (10) CN,
      (11) NO₂,
      (12) N(R^{A})R^{B},
      (13) C(O)N(R^{A})R^{B},
      (14) C(O)R^{A},
      (15) C(O)—C₁₋₆ haloalkyl,
      (16) C(O)OR^{A},
      (17) OC(O)N(R^{A})R^{B},
      (18) SR^{A},
      (19) S(O)R^{A},
      (20) S(O)₂R^{A},
      (21) S(O)₂N(R^{A})R^{B},
      (22) N(R^{A})S(O)₂R^{B},
      (23) N(R^{A})S(O)₂N(R^{A})R^{B},
      (24) N(R^{A})C(O)R^{B},
      (25) N(R^{A})C(O)N(R^{A})R^{B},
      (26) N(R^{A})C(O)—C(O)N(R^{A})R^{B}, or
      (27) N(R^{A})CO₂R^{B}, and
   (ii) from zero to 2 substituents are each independently:
      (1) CycE,
      (2) AryE,
      (3) O-AryE,
      (4) HetE,
      (5) HetF, or
      (6) C₁₋₆ alkyl substituted with CycE, AryE, O-AryE, HetE, O-HetE, or HetF;
HetR is (i) a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂ or (ii) a 6- to 10-membered saturated or mono-unsaturated, bridged or fused heterobicyclic ring containing from 1 to 4 heteroatoms independently selected from N, O and S, where each S is optionally oxidized to S(O) or S(O)₂; and wherein the saturated or mono-unsaturated heterocyclic or heterobicyclic ring is optionally substituted with a total of from 1 to 4 substituents, wherein:
   (i) from zero to 4 substituents are each independently halogen, CN, C₁₋₆ alkyl, OH, oxo, C(O)R^{A}, CO₂R^{A}, S(O)R^{A}, SR^{A}, S(O)₂R^{A}, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
   (ii) from zero to 2 substituents are each independently CycE, AryE, HetE, HetF, or C₁₋₆ alkyl substituted with CycE, AryE, HetE, or HetF;
R² is:
   (1) C₁₋₆ alkyl,
   (2) C₁₋₆ haloalkyl,
   (3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
   (3) CycB,
   (4) AryB,
   (5) HetB,
   (6) HetS,
   (7) C₁₋₆ alkyl substituted with CycB, AryB, HetB, or HetS,
   (8) N(R^{A})—C₁₋₆ alkyl,
   (9) N(R^{A})—C₁₋₆ alkyl, wherein the alkyl is substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
   (10) N(R^{A})-CycB,
   (11) N(R^{A})-AryB,
   (12) N(R^{A})-HetB, or
   (13) N(R^{A})—C₁₋₆ alkyl, wherein the alkyl is substituted with CycB, AryB, HetB, or HetS;
CycB independently has the same definition as CycA;
AryB independently has the same definition as AryA;
HetB independently has the same definition as HetA;
HetS independently has the same definition as HetR;
R³ is HetC, wherein HetC independently has the same definition as HetA;
R⁴ is H, C₁₋₆ alkyl, C(O)C₁₋₆ alkyl, C(O)-CycD, C(O)-AryD, C(O)-HetD, or C(O)HetU;
CycD independently has the same definition as CycA;
AryD independently has the same definition as AryA;
HetD independently has the same definition as HetA;
HetU independently has the same definition as HetR;
R⁵ is H or independently has the same definition as R¹;
each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
each CycE is independently C₃₋₈ cycloalkyl which is optionally substituted with a total of from 1 to 4 substituents, wherein:
   (i) from zero to 4 substituents are each independently halogen, C₁₋₆ alkyl, OH, O—C₁₋₆ alkyl, C₁₋₆ haloalkyl, or O—C₁₋₆ haloalkyl, and
   (ii) from zero to 2 substituents are each independently CycG, AryG, HetG, HetH, or C₁₋₆ alkyl substituted with CycG, AryG, O-AryG, HetG, or HetH;
each AryE is independently phenyl or naphthyl, wherein the phenyl or naphthyl is optionally substituted with a total of from 1 to 5 substituents, wherein:

(i) from zero to 5 substituents are each independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B}, and

(ii) from zero to 2 substituents are each independently CycG, AryG, HetG, HetH, or C₁₋₆ alkyl substituted with CycG, AryG, O-AryG, HetG, or HetH;
each HetE is independently (i) a 5- or 6-membered heteroaromatic ring containing from 1 to 4 heteroatoms independently selected from N, O and S, wherein each N is optionally in the form of an oxide, or (ii) a 9- or 10-membered fused heterobicyclic ring selected from 2,3-dihydrobenzo-1,4-dioxinyl and benzo-i,3-dioxolyl; and wherein the heteroaromatic ring or the heterobicyclic ring is optionally substituted with a total of from 1 to 4 substituents wherein:

(i) from zero to 4 substituents are each independently halogen, C₁₋₆ alkyl, C₁₋₆ haloalkyl, O—C₁₋₆ alkyl, O—C₁₋₆ haloalkyl, OH, C(O)R^{A}, CO₂R^{A}, SO₂R^{A}, N(R^{A})R^{B}, N(R^{A})C(O)N(R^{A})R^{B}, or N(R^{A})CO₂R^{B}, and

(ii) from zero to 2 substituents are each independently CycG, AryG, HetG, HetH, or C₁₋₆ alkyl substituted with CycG, AryG, O-AryG, HetG, or HetH;
each HetF is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, and wherein the saturated or mono-unsaturated heterocyclic ring is optionally substituted with a total of from 1 to 4 substituents, wherein:

(i) from zero to 4 substituents are each independently halogen, CN, C₁₋₆ alkyl, OH, oxo, O—C₁₋₆ alkyl, C₁₋₆ haloalkyl, O—C₁₋₆ haloalkyl, C(O)R^{A}, CO₂R^{A}, or SO₂R^{A}, and

(ii) from zero to 2 substituents are each independently CycG, AryG, HetG, HetH, or C₁₋₆ alkyl substituted with CycG, AryG, O-AryG, HetG, or HetH;
each CycG is independently C₃₋₈ cycloalkyl which is optionally substituted with from 1 to 4 substituents, each of which is independently halogen, C₁₋₆ alkyl, OH, O—C₁₋₆ alkyl, C₁₋₆ haloalkyl, or O—C₁₋₆ haloalkyl;
each AryG is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
each HetG is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, OH, C(O)R^{A}, CO₂R^{A}, SO₂R^{A}, N(R^{A})R^{B}, N(R^{A})C(O)N(R^{A})R^{B}, or N(R^{A})CO₂R^{B};

each HetH is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, and wherein the saturated or mono-unsaturated heterocyclic ring is optionally substituted with from 1 to 4 substituents, each of which is independently halogen, CN, C₁₋₆ alkyl, OH, oxo, O—C₁₋₆ alkyl, C₁₋₆ haloalkyl, O—C₁₋₆ haloalkyl, C(O)R^{A}, CO₂R^{A}, or SO₂R^{A};
each R^{A} is independently H or C₁₋₆ alkyl; and
each R^{B} is independently H or C₁₋₆ alkyl;
and with the proviso that:

(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

Other embodiments, aspects and features of the present invention are either further described in or will be apparent from the ensuing description, examples and appended claims.

### DETAILED DESCRIPTION OF THE INVENTION

The compounds of Formula I above, and pharmaceutically acceptable salts thereof, are HIV reverse transcriptase inhibitors. The compounds are useful for inhibiting HIV reverse transcriptase and for inhibiting HIV replication in vitro and in vivo. More particularly, the compounds of Formula I inhibit the polymerase function of HIV-1 reverse transcriptase. Based upon the testing of representative compounds of the invention in the assay set forth in Example 39 below, it is known that compounds of Formula I inhibit the RNA-dependent DNA polymerase activity of HIV-1 reverse transcriptase. Certain of the compounds of the present invention can also exhibit activity against drug resistant forms of HIV (e.g., mutant strains of HIV in which reverse transcriptase has a mutation at lysine 103 asparagine (K103N) and/or tyrosine 181→cysteine (Y181C)), and thus can exhibit decreased cross-resistance against currently approved antiviral therapies.

A first embodiment of the present invention is a compound of Formula I, or a pharmaceutically acceptable salt thereof, wherein each of the variables is as originally defined above (i.e., as defined in the Summary of the Invention); and with the proviso that:

(A) when R¹ is halogen, R² is AryB and AryB is unsubstituted phenyl or phenyl substituted with from 1 to 5 substituents each of which is independently halogen, NO₂, CN, C₁₋₆ alkyl, O—C₁₋₆ alkyl, C₁₋₆ alkylene-N(R^{A})R^{B}, S(O)₂N(R^{A})R^{B}, or C₁₋₆ haloalkyl, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently N(R^{A})R^{B}, C₁₋₆ alkyl, C₁₋₆ alkylene-N(R^{A})R^{B}, halogen, S(O)₂N(R^{A})R^{B}, CN, CycE, or C₁₋₆ haloalkyl or (ii) a bicyclic ring which is a 5-membered heteroaromatic ring containing from 1 to 2 N atoms that is fused with a cyclohexyl or cycloheptyl ring, wherein the bicyclic ring is attached to the rest of the molecule via an atom in the heteroaromatic ring.

A second embodiment of the present invention is a compound of Formula I, or a pharmaceutically acceptable salt thereof, wherein each of the variables is as originally defined above; and with the proviso that:

(A) when R¹ is halogen, and R² is AryB, then AryB is not unsubstituted phenyl or substituted phenyl.

A third embodiment of the present invention is a compound of Formula I, or a pharmaceutically acceptable salt thereof, wherein each of the variables is as originally defined above; proviso A as originally set forth above is applied; and any one or more of the following provisos are also applied:

(B) when R² is AryB, then AryB is not phenyl that is di-substituted or tri-substituted with OCH₃,

(C) when R⁵ is attached to the 6-position of the indole ring and is O—C₁₋₆ alkyl (e.g., methoxy), then R¹ is not oxazol-5-yl,

(D) when R¹ is (1) halogen, (2) CN, (3) C(O)R^{A}, (4) C(O)OR^{A}, (5) C(O)N(R^{A})R^{B}, (6) S(O)₂R^{A}, (7) S(O)₂N(R^{A})R^{B}, (8) N(R^{A})R^{B}, (9) C₁₋₆ alkyl, (10) C₁₋₆ haloalkyl, (11) C₂₋₆ alkenyl, (12) C₂₋₆ allcynyl, (13) OH, (14) O—C₁₋₆ alkyl, (15) O—C₁₋₆ haloalkyl, (16) C₁₋₆ alkyl substituted with OH, O—C₁₋₆ alkyl, O—C₁₋₆ haloalkyl, CN, N(R^{A})R^{B}, C(O)N(R^{A})R^{B}, C(O)R^{A}, CO₂R^{A}, or OC(O)N(R^{A})R^{B}, (17) CycA, (18) AryA, (19) HetA, (20) HetR, (21) C₁₋₆ alkyl substituted with CyA, AryA, HetA, or HetR, (22) J-CycA, (23) J-AryA, (24) J-HetA, or (25) J-HetR, R⁵ is H or independently has the same definition as R¹, and R² is other than CycB, AryB, HetB, or HetS that is attached to the rest of the molecule at a ring carbon atom, then R³ is not unsubstituted indazol-3-yl or substituted indazol-3-yl,

(E) when R¹ is CH₂-AryA or J-AryA, J in the definition of R¹ is O, S, S(O), S(O)₂, NH, or N(C₁₋₄ alkyl), and R⁵ is H, OH, halogen, CN, NO₂, C₁₋₄ alkyl, N(R^{A})R^{B}, N(R^{A})-CycA, N(R^{A})—CH₂-phenyl, N(R^{A})-phenyl, wherein either of the phenyl groups is optionally substituted with a total of from 1 to 5 substituents wherein (i) from zero to 5 substituents are each independently halogen, OH, NH², CO₂H, O—C₁₋₄ alkyl, C(O)O—C₁₋₄ alkyl, NHC(O)O—C₁₋₄ alkyl, and (ii) from zero to 2 substituents are each independently HetE, HetF, or phenyl optionally substituted by halogen or OH, then AryA in the definition of R¹ is not a di- or tri-substituted phenyl in which (i) one substituent in the di-substituted phenyl or each of two substituents in the tri-substituted phenyl is independently halogen, CN, C₁₋₆ alkyl, CF₃, CHF₂, CH₂F, or C₃₋₇ cycloalkyl, wherein either the one substituent on the di-substituted phenyl or one or both of the two substituents in the tri-substituted phenyl is ortho to the CH₂ or J moiety linking AryA to the rest of the molecule and (ii) the other substituent in the di- or tri-substituted phenyl is OC(O)N(R^{A})R^{B}, S(O)₂R^{A}, S(O)₂N(R^{A})R^{B}, N(R^{A})S(O)₂R^{B}, N(R^{A})S(O)₂N(R^{A})R^{B}, N(R^{A})C(O)R^{B}, N(R^{A})C(O)N(R^{A})R^{B}, N(R^{A})CO₂R^{B}, HetE, HetF, (CH₂)₁₋₂-HetE, or (CH₂)₁₋₂-HetF;

(F) when R¹ is CH₂CH₂-HetA or J-HetA, J in the definition of R¹ is OCH₂, SCH₂, or S(O)₂CH₂, and HetA in the definition of R¹ is (i) a 5- or 6-membered heteroaromatic ring containing from 1 to 3 N atoms wherein the ring is optionally mono- or di-substituted, (ii) a 5-membered heteroaromatic ring containing one O or S atom and from zero to 2 N atoms, wherein the ring is optionally mono- or di-substituted, or (iii) an 8- to 10-membered aromatic bicyclic, fused ring system containing from 1 to 3 N atoms, wherein the ring system is optionally mono- or di-substituted, then R³ is not 1H-tetrazol-5-yl or 2H-tetrazol-5-yl, and

(G) when R¹ is CH₂CH₂-AryA or J-AryA, J in the definition of R¹ is OCH₂, SCH₂, or S(O)₂CH₂, and AryA in the definition of R¹ is an aryl other than phenyl, wherein the aryl other than phenyl is optionally mono- or di-substituted, then R³ is not 1H-tetrazol-5-yl or 2H-tetrazol-5-yl.

A fourth embodiment of the present invention is identical to the third embodiment, except that proviso B is as follows:

(B) (i) when R² is AryB, then AryB is not an aryl that is di-substituted or tri-substituted with O—C₁₋₆ alkyl or (ii) when R² is HetB, then HetB is not a heteroaryl that is di-substituted or tri-substituted with O—C₁₋₆ alkyl.

A fifth embodiment of the present invention is a compound of Formula I, or a pharmaceutically acceptable salt thereof, wherein each of the variables is as originally defined above; proviso A as set forth in the first embodiment is applied; and any one or more of the following provisos are also applied:

(B) (i) when R² is AryB, then AryB is not an aryl that is di-substituted or tri-substituted with O—C₁₋₆ alkyl or (ii) when R² is HetB, then HetB is not a heteroaryl that is di-substituted or tri-substituted with O—C₁₋₆ alkyl,

(C) when R⁵ is attached to the 6-position of the indole ring and is (1) halogen, (2) C₁₋₆ alkyl, (3) C₁₋₆ haloalkyl, (4) O—C₁₋₆ alkyl, (5) O—C₁₋₆ haloalkyl, (6) O-CycA, (7) O-AryA, (8) O-HetA, (9) O-HetR, (10) C₁₋₆ alkyl substituted with OH, O—C₁₋₆ alkyl, O—C₁₋₆ haloalkyl, CN, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, or (11) C₁₋₆ alkyl substituted with CycA, AryA, HetA, or HetR, then R¹ is not unsubstituted oxazolyl or oxazolyl substituted with 1 or 2 substituents each of which is independently (1) halogen, (2) CN, (3) C₁₋₆ alkyl, (4) C₁₋₆ haloalkyl, (5) C₁₋₆ alkyl substituted with OH, O—C₁₋₆ alkyl, O—C₁₋₆ haloalkyl, CN, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, (6) C₁₋₆ alkyl substituted with 2 to 3 OH, (7) C₁₋₆ substituted with CycE, AryE, O-AryE, HetE, O-HetE, or HetF (8) OH, (9) O—C₁₋₆ alkyl, (10) O—C₁₋₆ haloalkyl, or (11) O-AryE,

(D) when R¹ is (1) halogen, (2) CN, (3) C(O)R^{A}, (4) C(O)OR^{A}, (5) C(O)N(R^{A})R^{B}, (6) S(O)₂R^{A}, (7) S(O)₂N(R^{A})R^{B}, (8) N(R^{A})R^{B}, (9) C₁₋₆ alkyl, (10) C₁₋₆ haloalkyl, (11) C₂₋₆ alkenyl, (12) C₂₋₆ alkynyl, (13) OH, (14) O—C₁₋₆ alkyl, (15) O—C₁₋₆ haloalkyl, (16) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, (17) CycA, (18) AryA, (19) HetA, (20) HetR, (21) C₁₋₆ alkyl substituted with CycA, AryA, HetA, or HetR, (22) J-CycA, (23) J-AryA, (24) J-HetA, or (25) J-HetR, R⁵ is H or independently has the same definition as R¹, and R² is other than CycB, AryB, HetB, or HetS that is attached to the rest of the molecule at a ring carbon atom, then R³ is not an unsubstituted or substituted heteroaryl selected from the group consisting of

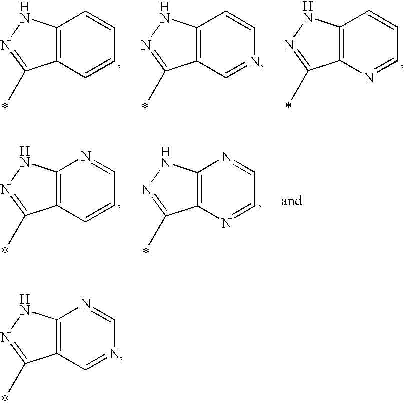

(E) when R¹ is C₁₋₆ alkylene-AryA or J-AryA, J in the definition of R¹ is O, S, S(O), S(O)₂, or N(R^{A}), and R⁵ is H, OH, halogen, CN, NO₂, C₁₋₆ alkyl, N(R^{A})R^{B}, N(R^{A})-CycA, N(R^{A})—C₁₋₆ alkylene-AryA, N(R^{A})-AryA, then AryA in the definition of R¹ is not a di- or tri-substituted phenyl in which (i) one substituent in the di-substituted phenyl or each of two substituents in the tri-substituted phenyl is independently halogen, CN, C₁₋₆ alkyl, CF₃, CHF₂, CH₂F, or C₃₋₇ cycloalkyl, wherein either the one substituent on the di-substituted phenyl or one or both of the two substituents in the tri-substituted phenyl is ortho to the C₁₋₆ alkylene or J moiety linking AryA to the rest of the molecule and (ii) the other substituent in the di- or tri-substituted phenyl is OC(O)N(R^{A})R^{B}, S(O)₂R^{A}, S(O)₂N(R^{A})R^{B}, N(R^{A})S(O)₂R^{B}, N(R^{A})S(O)₂N(R^{A})R^{B}, N(R^{A})C(O)R^{B}, N(R^{A})C(O)N(R^{A})R^{B}, N(R^{A})CO₂R^{B}, HetE, HetF, C₁₋₆ alkylene-HetE, or C₁₋₆ alkylene-HetF,

(F) when R¹ is CH₂CH₂-HetA or J-HetA, J in the definition of R¹ is OCH₂, SCH₂, or S(O)₂CH₂, then R³ is not tetrazolyl, and

(G) when R¹ is CH₂CH₂-AryA or J-AryA, J in the definition of Ri is OCH₂, SCH₂, or S(O)₂CH₂, then R³ is not tetrazolyl.

A sixth embodiment of the present invention is identical to the fifth embodiment, except that proviso C is as follows:

(C) when R⁵ is attached to the 6-position of the indole ring and is (1) halogen, (2) C₁₋₆ alkyl, (3) C₁₋₆ haloalkyl, (4) O—C₁₋₆ alkyl, (5) O—C₁₋₆ haloalkyl, (6) O-CycA, (7) O-AryA, (8) O-HetA, (9) O-HetR, (10) C₁₋₆ alkyl substituted with OH, O—C₁₋₆ alkyl, O—C₁₋₆ haloalkyl, CN, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B}, or (11) C₁₋₆ alkyl substituted with CycA, AryA, HetA, or HetR, then R¹ is not unsubstituted oxazolyl or substituted oxazolyl.

A seventh embodiment of the present invention is a compound of Formula I, or a pharmaceutically acceptable salt thereof, wherein each of the variables is as originally defined above; proviso A as set forth in the second embodiment is applied; and any one or more of the following provisos are also applied:

(B) (i) when R² is AryB, then AryB is not an aryl that is di-substituted or tri-substituted with O—C₁₋₆ alkyl or (ii) when R² is HetB, then HetB is not a heteroaryl that is di-substituted or tri-substituted with O—C₁₋₆ alkyl,

(C) when R⁵ is attached to the 6-position of the indole ring and is other than H, then R¹ is not unsubstituted oxazolyl or substituted oxazolyl,

(D) when R² is other than CycB, AryB, HetB, or HetS that is attached to the rest of the molecule at a ring carbon atom, then R³ is not an unsubstituted or substituted heteroaryl selected from the group consisting of

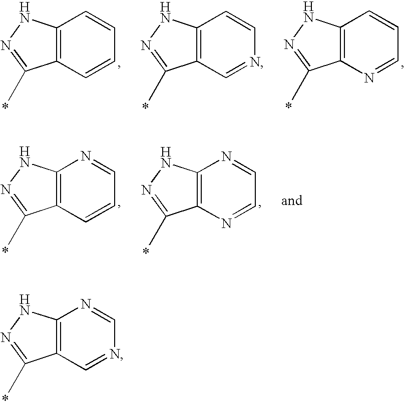

(E) when R¹ is C₁₋₆ alkylene-AryA or J-AryA, and J is O, S, S(O), S(O)₂, or N(R^{A}), then AryA in the definition of R¹ is not a di- or tri-substituted phenyl in which at least one of the substituents in the di -or tri-substituted phenyl is ortho to the C₁₋₆ alkylene or J moiety linking AryA to the rest of the molecule,

(F) when R¹ is C₁₋₆ alkylene-HetA or J-HetA, then R³ is not tetrazolyl, and

(G) when R¹ is C₁₋₆ alkylene-AryA or J-AryA, then R³ is not tetrazolyl.

An eighth embodiment of the present invention is identical to the seventh embodiment, except that proviso D is as follows:

(D) R³ is not an unsubstituted or substituted indazol-3-yl.

A ninth embodiment of the present invention is identical to the seventh embodiment, except that proviso D is as follows:

(D) R³ is not an unsubstituted or substituted heteroarvl selected from the grout) consisting of

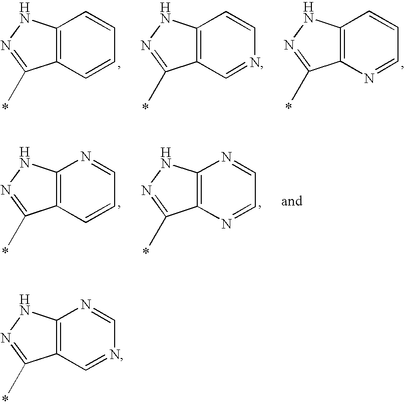

A tenth embodiment of the present invention is a compound of Formula I, or a pharmaceutically acceptable salt thereof, wherein R¹ is:
(1) halogen,
(2) CN,
(3) NO₂,
(4) N(R^{A})R^{B},
(5) N(R^{A})S(O)₂R^{B},
(6) N(R^{A})C(O)R^{B},
(7) C₁₋₆ alkyl,
(8) C₁₋₆ haloalkyl,
(9) C₂₋₆ alkenyl,
(10) OH,
(11) O—C₁₋₆ alkyl,
(12) O—C₁₋₆ haloalkyl,
(13) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(14) CycA,
(15) AryA,
(16) HetA, or
(17) C₁₋₆ alkyl substituted with CycA, AryA, or HetA; and
   R⁵ is H; and all other variables are as originally defined; and with the proviso that:

(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

A first aspect of the tenth embodiment is a compound of Formula I, or a pharmaceutically acceptable salt thereof, wherein the compound is as defined in the tenth embodiment, except that it incorporates proviso A as set forth in the first embodiment. A second aspect of the tenth embodiment is a compound of Formula I, or a pharmaceutically acceptable salt thereof, wherein the compound is as defined in the tenth embodiment, except that it incorporates proviso A as set forth in the second embodiment. A third aspect of the tenth embodiment is a compound of Formula I, or a pharmaceutically acceptable salt thereof, wherein the compound is as defined in the tenth embodiment, except that it incorporates the provisos set forth in the third embodiment; i.e., proviso A as originally set forth above is applied; and any one or more of provisos B to G as set forth in the third embodiment are also applied. A fourth aspect of the tenth embodiment is identical to the third aspect, except that proviso B is as set forth in the fourth embodiment. A fifth aspect of the tenth embodiment is a compound of. Formula I, or a pharmaceutically acceptable salt thereof, wherein the compound is as defined in the tenth embodiment, except that it incorporates the provisos set forth in the fifth embodiment; i.e., proviso A as set forth in the first embodiment is applied; and any one or more of provisos B to G as set forth in the fifth embodiment are also applied. A sixth aspect of the tenth embodiment is identical to the fifth aspect, except that proviso C is as set forth in the sixth embodiment. A seventh aspect of the tenth embodiment is a compound of Formula I, or a pharmaceutically acceptable salt thereof, wherein the compound is as defined in the tenth embodiment, except that it incorporates the provisos set forth in the seventh embodiment; i.e., proviso A as set forth in the second embodiment is applied; and any one or more of provisos B to G as set forth in the seventh embodiment are also applied. An eighth aspect of the tenth embodiment is identical to the seventh aspect, except that proviso D is as set forth in the eighth embodiment. A ninth aspect of the tenth embodiment is identical to the seventh aspect, except that proviso D is as set forth in the ninth embodiment.

It is understood that the provisos set forth in the foregoing aspects of the tenth embodiment can be modified to conform with the definitions of the variables set forth in the tenth embodiment. For example, in view of the definition of R¹ in the tenth embodiment, proviso D in the third aspect can be modified to read as follows:

(D) when R¹ is (1) halogen, (2) CN, (3) N(R^{A})R^{B}, (4) C₁₋₆ alkyl, (5) C₁₋₆ haloalkyl, (6) C₂₋₆ alkenyl, (7) OH, (8) O—C₁₋₆ alkyl, (9) O—C₁₋₆ haloalkyl, (10) C₁₋₆ alkyl substituted with OH, O—C₁₋₆ alkyl, O—C₁₋₆ haloalkyl, CN, N(R^{A})R^{B}, C(O)N(R^{A})R^{B, C(O)R}^{A}, CO₂R^{A}, S(O)₂N(R^{A})R^{B}, or OC(O)N(R^{A})R^{B}, (11) CycA, (12) AryA, (13) HetA, or (14) C₁₋₆ alkyl substituted with CycA, AryA, or HetA, and R² is other than CycB, AryB, HetB, or HetS that is attached to the rest of the molecule at a ring carbon atom, then R³ is not unsubstituted indazol-3-yl or substituted indazol-3-yl.

As another example, since the variable J-HetA is not included in the definition of R¹ in the tenth embodiment, proviso F in the third and fifth and seventh aspects can be modified to remove the language directed to J-HetA. Similarly, since J-AryA is not included in the definition of R¹ in the tenth embodiment, provisos E and Gin the third and fifth and seventh aspects can be modified to remove the language directed to J-AryA.

An eleventh embodiment of the present invention is a compound of Formula I, or a pharmaceutically acceptable salt thereof, wherein R¹ is Cl, Br, or F; R⁵ is H; and all other variables are as originally defined; and with the proviso that:

(A) when R² is AryB and AryB is unsubstituted phenyl or phenyl substituted with froml to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, and R⁴ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

A first aspect of the eleventh embodiment is a compound of Formula I, or a pharmaceutically acceptable salt thereof, wherein the compound is as defined in the eleventh embodiment, except that it incorporates proviso A as set forth in the first embodiment. A second aspect of the eleventh embodiment is a compound of Formula I, or a pharmaceutically acceptable salt thereof, wherein the compound is as defined in the eleventh embodiment, except that it incorporates proviso A as set forth in the second embodiment. A third aspect of the eleventh embodiment is a compound of Formula I, or a pharmaceutically acceptable salt thereof, wherein the compound is as defined in the eleventh embodiment, except that it incorporates the applicable provisos set forth in the third embodiment; i.e., proviso A as originally set forth above is applied; and either one or both of provisos B and D as set forth in the third embodiment are also applied, wherein these provisos can be modified to read as follows in conformance with the definitions of the variables set forth in the eleventh embodiment:

(B) when R² is AryB, then AryB is not phenyl that is di-substituted or tri-substituted with OCH₃, and

(D) when R² is other than CycB, AryB, HetB, or HetS that is attached to the rest of the molecule at a ring carbon atom, then R³ is not unsubstituted indazol-3-yl or substituted indazol-3-yl.

A fourth aspect of the eleventh embodiment is identical to the third aspect, except that proviso B is as set forth in the fourth embodiment. A fifth aspect of the eleventh embodiment is a compound of Formula I, or a pharmaceutically acceptable salt thereof, wherein the compound is as defined in the eleventh embodiment, except that it incorporates the applicable provisos set forth in the fifth embodiment; i.e., proviso A as set forth in the first embodiment is applied; and any one or more of provisos B and D as set forth in the fifth embodiment are also applied. These provisos can be modified to read as follows:

(B) (i) when R² is AryB, then AryB is not an aryl that is di-substituted or tri-substituted with O—C₁₋₆ alkyl or (ii) when R² is HetB, then HetB is not a heteroaryl that is di-substituted or tri-substituted with O—C₁₋₆ alkyl,

(D) when R² is other than CycB, AryB, HetB, or HetS that is attached to the rest of the molecule at a ring carbon atom, then R³ is not an unsubstituted or substituted heteroaryl selected from the group consisting of

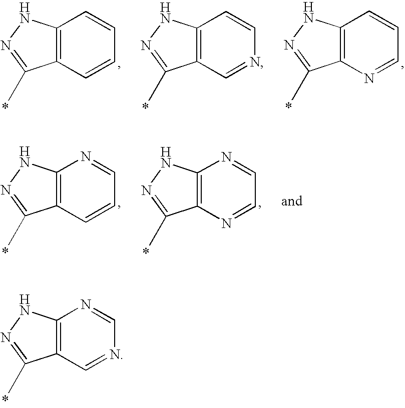

A fifth aspect of the eleventh embodiment is identical to the fourth aspect, except that proviso D is as set forth in the eighth embodiment. A sixth aspect of the eleventh embodiment is identical to the fourth aspect, except that proviso D is as set forth in the ninth embodiment.

A twelfth embodiment of the present invention is a compound of Formula I, or a pharmaceutically acceptable salt thereof, wherein R² is AryB or HetS; and all other variables are as originally defined; and with the proviso that:

(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

A first aspect of the twelfth embodiment is a compound of Formula I, or a pharmaceutically acceptable salt thereof, wherein the compound is as defined in the twelfth embodiment, except that it incorporates proviso A as set forth in the first embodiment. A second aspect of the twelfth embodiment is a compound of Formula I, or a pharmaceutically acceptable salt thereof, wherein the compound is as defined in the twelfth embodiment, except that it incorporates proviso A as set forth in the second embodiment. A third aspect of the twelfth embodiment is a compound of Formula I, or a pharmaceutically acceptable salt thereof, wherein the compound is as defined in the twelfth embodiment, except that it incorporates the provisos set forth in the third embodiment; i.e., proviso A as originally set forth above is applied; and any one or more of provisos B to G as set forth in the third embodiment are also applied. A fourth aspect of the twelfth embodiment is identical to the third aspect, except that proviso B is as set forth in the fourth embodiment. A fifth aspect of the twelfth embodiment is a compound of Formula I, or a pharmaceutically acceptable salt thereof, wherein the compound is as defined in the twelfth embodiment, except that it incorporates the provisos set forth in the fifth embodiment; i.e., proviso A as set forth in the first embodiment is applied; and any one or more of provisos B to G as set forth in the fifth embodiment are also applied. A sixth aspect of the twelfth embodiment is identical to the fifth aspect, except that proviso C is as set forth in the sixth embodiment.

A seventh aspect of the twelfth embodiment is a compound of Formula I, or a pharmaceutically acceptable salt thereof, wherein the compound is as defined in the twelfth embodiment, except that it incorporates the provisos set forth in the seventh embodiment; i.e., proviso A as set forth in the second embodiment is applied; and any one or more of provisos B to G as set forth in the seventh embodiment are also applied. An eighth aspect of the twelfth embodiment is identical to the seventh aspect, except that proviso D is as set forth in the eighth embodiment. A ninth aspect of the twelfth embodiment is identical to the seventh aspect, except that proviso D is as set forth in the ninth embodiment.

It is understood that the provisos set forth in the foregoing aspects of the twelfth embodiment can be modified to conform with the definitions of the variables set forth in the twelfth embodiment. For example, in view of the definition of R² in the twelfth embodiment, proviso D as set forth in the third, fourth, fifth, sixth and seventh aspects places no restriction on the scope of the embodiment and need not be included in the proviso.

A thirteenth embodiment of the present invention is a compound of Formula I, or a pharmaceutically acceptable salt thereof, wherein AryB is phenyl, wherein the phenyl is optionally substituted with a total of from 1 to 5 substituents, each of which is independently:
(1) C₁₋₄ alkyl,
(2) O—C₁₋₄ alkyl,
(3) C₁₋₄ haloalkyl,
(4) O—C₁₋₄ haloalkyl,
(5) OH,
(6) halogen,
(7) CN,
(8) NO₂,
(9) NH₂,
(10) N(H)—C₁₋₄ alkyl,
(11) N(C₁₋₄ alkyl)₂,
(12) C(O)NH₂,
(13) C(O)N(H)—C₁₋₄ alkyl,
(14) C(O)N(C₁₋₄ alkyl)₂,
(15) C(O)—C₁₋₄ alkyl,
(16) CO₂—C₁₋₄ alkyl,
(17) S—C₁₋₄ alkyl,
(18) S(O)—C₁₋₄ alkyl,
(19) SO₂—C₁₋₄ alkyl,
(20) SO₂NH₂,
(21) SO₂N(H)—C₁₋₄ alkyl,
(22) SO₂N(C₁₋₄ alkyl)₂,
(23) SO₂N(H)C(O)—C₁₋₄ alkyl,
(24) SO₂N(C₁₋₄ alkyl)C(O)—C₁₋₄ alkyl,
(25) N(H)C(O)—C₁₋₄ alkyl, or
(26) N(C₁₋₄ alkyl)C(O)—C₁₋₄ alkyl; and
   HetS is a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring contains a nitrogen atom which is directly attached to the rest of the molecule and optionally contains an additional heteroatom selected from N, O, and S, where the S is optionally oxidized to S(O) or S(O)₂; and wherein the heterocyclic or heterobicyclic ring is optionally substituted with a total of from 1 to 4 substituents, wherein:
(i) from zero to 4 substituents are each independently Cl, Br, F, C₁₋₄alkyl, OH, oxo, S(O)₂—C₁₋₄ alkyl, O—C₁₋₄ alkyl, O—C₁₋₄ haloalkyl, or C₁₋₄ haloalkyl; and
(ii) from zero to 1 substituent is AryE, HetE, CH₂-AryE, or CH₂-HetE;
   and all other variables are as defined in the twelfth embodiment; and with the proviso that:

(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, SO₂NH₂, or C₁₋₄ haloalkyl having from 1 to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

The thirteenth embodiment has nine aspects corresponding to the nine aspects of the twelfth embodiment as set forth above, wherein it is understood that the provisos set forth in these aspects can be modified to conform with the definitions of the variables set forth in the thirteenth embodiment.

A fourteenth embodiment of the present invention is a compound of Formula I, or a pharmaceutically acceptable salt thereof, wherein R³ is HetC; and HetC is:
(i) a 5-membered heteroaromatic ring containing from 1 to 3 heteroatoms independently selected from 1 to 3 N atoms, from zero to 1 O atom, and from zero to 1 S atom, wherein the heteroaromatic ring is connected to the rest of the molecule via a ring carbon, and the heteroaromatic ring is optionally substituted with from 1 to 2 substituents each of which is independently
   (1) C₁₋₄ alkyl,
   (2) C₁₋₄ alkyl substituted with OH or O—C₁₋₄ alkyl,
   (3) C₁₋₄ alkyl substituted with from 2 to 4 OH,
   (4) O—C₁₋₄ alkyl,
   (5) C₁₋₄ haloalkyl,
   (6) O—C₁₋₄ haloalkyl,
   (7) OH,
   (8) Cl, Br, or F,
   (9) CN,
   (10) C(O)N(H)—C₁₋₄ alkyl,
   (11) C(O)N(C₁₋₄ alkyl)₂,
   (12) S(O)₂—C₁₋₄ alkyl,
   (13) S(O)₂ NH₂,
   (14) S(O)₂N(H)—C₁₋₄ alkyl,
   (15) S(O)₂N(C₁₋₄ alkyl)₂,
   (16) CycE, AryE, or HetE, or
   (17) CH₂-CycE, CH₂-AryE, CH₂—O-AryE, or CH₂-HetE, or
(ii) 5-membered heteroaromatic ring containing from 1 to 2 heteroatoms independently selected from 1 to 2 N atoms, from zero to 1 O atom, and from zero to 1 S atom, wherein the heteroaromatic ring is connected to the rest of the molecule via a ring carbon and has fused thereto a benzene ring wherein the benzene ring is optionally substituted with from 1 to 3 substituents each of which is independently
(1) C₁₋₄ alkyl,
(2) O—C₁₋₄ alkyl,
(3) C₁₋₄ haloalkyl,
(4) O—C₁₋₄ haloalkyl,
(5) OH,
(6) Cl, Br, or F,
(7) CN,
(8) C(O)N(H)—C₁₋₄ alkyl,
(9) C(O)N(C₁₋₄ alkyl)₂,
(10) S(O)₂—C₁₋₄ alkyl,
(11) S(O)₂NH₂,
(12) S(O)₂N(H)—C₁₋₄ alkyl, or
(13) S(O)₂N(C₁₋₄ alkyl)₂;
   and all other variables are as originally defined above; and with the proviso that:

(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not a 5-membered heteroaromatic ring containing from 1 to 3 heteroatoms independently selected from 1 to 3 N atoms, from zero to 1 O atom, and from zero to 1 S atom, wherein the heteroaromatic ring is connected to the rest of.the molecule via a ring carbon and wherein the heteroaromatic ring is unsubstituted or substituted with one or more substituents each of which is independently C₁₋₄ alkyl, Cl, Br, F, S(O)₂NH₂, CN, C₃₋₅ cycloalkyl, or C₁₋₄ haloalkyl having from 1 to 3 halogen substituents.

The fourteenth embodiment has nine aspects corresponding to the nine aspects of the twelfth embodiment as set forth above, wherein it is understood that the provisos set forth in these aspects can be modified to conform with the definitions of the variables set forth in the fourteenth embodiment.

A fifteenth embodiment of the present invention is a compound of Formula I, or a pharmaceutically acceptable salt thereof, wherein R³ is:
R³ is

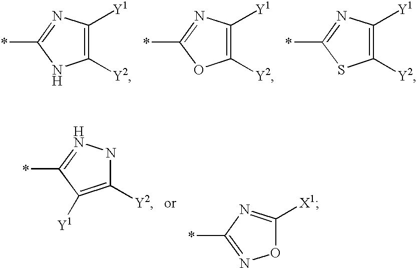

X¹ is:
   (1) H,
   (2) C₁₋₄ alkyl,
   (3) C₁₋₄ alkyl substituted with OH or O—C₁₋₄ alkyl,
   (4) C₁₋₄ alkyl substituted with from 2 to 4 OH,
   (5) C₃₋₆ cycloalkyl which is optionally substituted with C₁₋₄ alkyl or phenyl,
   (6) phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂,
   (7) phenyl substituted with a heterocyclic ring selected from the group consisting of:

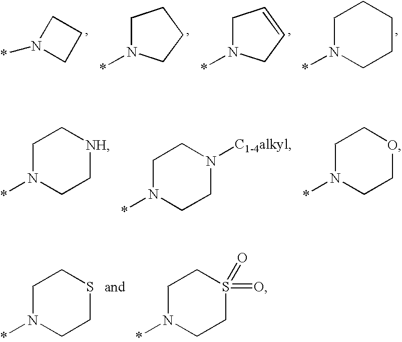

wherein the asterisk denotes the point of attachment to the rest of the molecule,
(8) CH₂-phenyl,
(9) CH₂—O-phenyl,
(10) heteroaryl selected from the group consisting of pyrrolyl, imidazolyl, furanyl, thienyl, oxazolyl, thiazolyl, pyridinyl, pyrimidinyl, and pyrazinyl, wherein the heteroaryl is optionally substituted with from 1 to 3 substituents each of which is independently Cl, Br, F, C₁₋₄ alkyl, CF₃, OH, O—C₁₋₄ alkyl, or OCF₃, or
(11) heteroaryl selected from the group consisting of 2,3-dihydrobenzo-1,4-dioxinyl and benzo-1,3-dioxolyl;
Y¹ independently has the same definition as X¹; and
Y² independently has the same definition as X¹;
or alternatively, Y¹ and Y² together with the carbon atoms to which each is attached form a benzo ring;
and all other variables are as defined in the fourteenth embodiment; and with the proviso that:

(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then (i) X¹ in the definition of R³ is not H, C₁₋₄ alkyl, or C₃₋₅ cycloalkyl and (ii) one of Y¹ and Y² in the definition of R³ is not H, C₁₋₄ alkyl, or C₃₋₅ cycloalkyl when the other of Y¹ and Y² is H, C₁₋₄ alkyl, or C₃₋₅ cycloalkyl.

The fifteenth embodiment has nine aspects corresponding to the nine aspects of the twelfth embodiment as set forth above, wherein it is understood that the provisos set forth in these aspects can be modified to conform with the definitions of the variables set forth in the fifteenth embodiment.

A sixteenth embodiment of the present invention is a compound of Formula I, or a pharmaceutically acceptable salt thereof, wherein R⁴ is H; and all other variables are as originally defined above; and with the proviso that:

(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituen and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

The sixteenth embodiment has nine aspects corresponding to the nine aspects of the twelfth embodiment as set forth above, wherein it is understood that the provisos set forth in these aspects can be modified to conform with the definitions of the variables set forth in the sixteenth embodiment.

A seventeenth embodiment of the present invention is a compound of Formula I, or a pharmaceutically acceptable salt thereof, wherein each R^{A} and R^{B} is independently —H or —C₁₋₄ alkyl; and all other variables are as originally defined or as defined in any one of the preceding embodiments or aspects thereof.

An eighteenth embodiment of the present invention is a compound of Formula I, or a pharmaceutically acceptable salt thereof, wherein each R^{A} and R^{B} is independently —H or methyl; and all other variables are as originally defined or as defined in any one of the preceding embodiments or aspects thereof.

A first class of the present invention includes compounds of Formula I and pharmaceutically acceptable salts thereof, wherein:
R¹ is halogen;
R² is:
   (i) phenyl, wherein the phenyl is optionally substituted with a total of from 1 to 3 substituents, each of which is independently:
      (1) C₁₋₄ alkyl,
      (2) O—C₁₋₄ alkyl,
      (3) C₁₋₄ haloalkyl,
      (4) O—C₁₋₄ haloalkyl,
      (5) OH,
      (6) halogen,
      (7) CN,
      (8) NO₂,
      (9) NH₂,
      (10) N(H)—C₁₋₄ alkyl,
      (11) N(C₁₋₄ alkyl)₂,
      (12) C(O)NH₂,
      (13) C(O)N(H)—C₁₋₄ alkyl,
      (14) C(O)N(C₁₋₄ alkyl)₂,
      (15) C(O)—C₁₋₄ alkyl,
      (16) CO₂—C₁₋₄ alkyl,
      (17) S—C₁₋₄ alkyl,
      (18) S(O)—C₁₋₄ alkyl,
      (19) SO₂—C₁₋₄ alkyl,
      (20) SO₂NH₂,
      (21) SO₂N(H)—C₁₋₄ alkyl,
      (22) SO₂N(C₁₋₄ alkyl)₂,
      (23) SO₂N(H)C(O)—C₁₋₄ alkyl,
      (24) SO₂N(C₁₋₄ alkyl)C(O)—C₁₋₄ alkyl,
      (25) N(H)C(O)—C₁₋₄ alkyl, or
      (26) N(C₁₋₄ alkyl)C(O)—C₁₋₄ alkyl, or
   (ii) HetS, wherein HetS is a 5- or 6-membered, saturated or mono-unsaturated heterocyclic ring containing a nitrogen atom that is directly attached to the rest of the molecule and optionally containing an additional heteroatom selected from N, O, and S, where the S is optionally oxidized to S(O) or S(O)₂; and wherein the heterocyclic ring is optionally substituted with a total of from 1 to 3 substituents, each of which is independently Cl, Br, F, C₁₋₄ alkyl, OH, oxo, S(O)₂—C₁₋₄ alkyl, O—C₁₋₄ alkyl, O—C₁₋₄ haloalkyl, or C₁₋₄ haloalkyl;
R³ is:
   (i) a 5-membered heteroaromatic ring containing from 1 to 3 heteroatoms independently selected from 1 to 3 N atoms, from zero to 1 O atom, and from zero to 1 S atom, wherein the heteroaromatic ring is connected to the rest of the molecule via a ring carbon, and the heteroaromatic ring is optionally substituted with from 1 to 2 substituents each of which is independently:
      (1) C₁₋₄ alkyl,
      (2) C₁₋₄ alkyl substituted with OH or O—C₁₋₄ alkyl,
      (3) C₁₋₄ alkyl substituted with from 2 to 4 OH,
      (4) O—C₁₋₄ alkyl,
      (5) C₁₋₄ haloalkyl,
      (6) O—C₁₋₄ haloalkyl,
      (7) OH,
      (8) Cl, Br, or F,
      (9) CN,
      (10) C(O)N(H)—C₁₋₄ alkyl,
      (11) C(O)N(C₁₋₄ alkyl)₂,
      (12) S(O)₂—C₁₋₄ alkyl,
      (13) S(O)₂NH₂,
      (14) S(O)₂N(H)—C₁₋₄ alkyl,
      (15) S(O)₂N(C₁₋₄ alkyl)₂,
      (16) CycE, AryE, or HetE, or
      (17) CH₂-CycE, CH₂-AryE, CH₂—O-AryE, or CH₂-HetE, or
   (ii) 5-membered heteroaromatic ring containing from 1 to 2 heteroatoms independently selected from 1 to 2 N atoms, from zero to 1 O atom, and from zero to 1 S atom, wherein the heteroaromatic ring is connected to the rest of the molecule via a ring carbon and has fused thereto a benzene ring wherein the benzene ring is optionally substituted with from 1 to 3 substituents each of which is independently
      (1) C₁₋₄ alkyl,
      (2) O—C₁₋₄ alkyl,
      (3) C₁₋₄ haloalkyl,
      (4) O—C₁₋₄ haloalkyl,
      (5) OH,
      (6) Cl, Br, or F,
      (7) CN,
      (8) C(O)N(H)—C₁₋₄ alkyl,
      (9) C(O)N(C₁₋₄ alkyl)₂,
      (10) S(O)₂—C₁₋₄ alkyl,
      (11) S(O)₂NH₂,
      (12) S(O)₂N(H)—C₁₋₄ alkyl, or
      (13) S(O)₂N(C₁₋₄ alkyl)₂;
each CycE is independently C₃.₆ cycloalkyl which is optionally substituted with a total of from 1 to 3 substituents, wherein:
   (i) from zero to 3 substituents are each independently C₁₋₄ alkyl, OH, or O—C₁₋₄ alkyl, and
   (ii) from zero to 1 substituent is phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂;
each AryE is independently phenyl, which is optionally substituted with a total of from 1 to 3 substituents, wherein:
   (i) from zero to 3 substituents are each independently C₁₋₄ alkyl, O—C₁₋₄ alkyl, C₁₋₄ fluoroalkyl, O—C₁₋₄ fluoroalkyl, OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂N alkyl, or S(O)₂N(C₁₋₄ alkyl)₂, and
   (ii) from zero to 1 substituent is a 4- to 7-membered saturated or mono-unsaturated heterocyclic ring containing from 1 to 2 heteroatoms selected from 1 to 2 N atoms, zero to 1 O atom, and zero to 1 S atom, where the S is optionally oxidized to S(O) or S(O)₂, and wherein the saturated or mono-unsaturated heterocyclic ring is optionally substituted with from 1 to 3 substituents, each of which is independently C₁₋₄ alkyl, OH, oxo, O—C₁₋₄ alkyl, C(O)—C₁₋₄ alkyl, C(O)O—C₁₋₄ alkyl, or SO₂—C₁₋₄ alkyl;
each HetE is independently (i) a 5- or 6-membered heteroaromatic ring selected from the group consisting of pyrrolyl, pyrazolyl, imidazolyl, triazolyl, tetrazolyl, furanyl, oxazolyl, isoxazolyl, thienyl, thiazolyl, isothiazolyl, oxadiazolyl, pyridinyl, pyrimidinyl, and pyrazinyl or (ii) a 9- or 10-membered fused heterobicyclic ring selected from 2,3-dihydrobenzo-1,4-dioxinyl and benzo-1,3-dioxolyl; and wherein the heteroaromatic ring or the heterobicyclic ring is optionally substituted with a total of from 1 to 3 substituents each of which is independently halogen, C₁₋₄ alkyl, C₁₋₄ fluoroalkyl, O—C₁₋₄ alkyl, O—C₁₋₄ fluoroalkyl, or OH;
R⁴ is H; and
R⁵ is H;
and with the proviso that:

(A) when R² is unsubstituted phenyl or phenyl substituted with from 1 to 3 substituents each of which is independently halogen, NO₂, CN, C₁₋₄ alkyl, O—C₁₋₄ alkyl, SO₂NH₂, or C₁₋₄ haloalkyl having from 1 to 3 halogen substituents, then R³ is not a 5-membered heteroaromatic rin containing from 1 to 3 heteroatoms selected from 1 to 3 N atoms, from zero to 1 O atom, and from zero to 1 S atom, wherein the heteroaromatic ring is connected to the rest of the molecule via a ring carbon, and the heteroaromatic ring is unsubstituted or substituted with from 1 to 2 substituents each of which is independently C₁₋₄ alkyl, Cl, Br, F, SO₂NH₂, CN, C₃₋₅ cycloalkyl, or C₁₋₄ haloalkyl having from 1 to 3 halogen substituents.

A first sub-class of the first class includes compounds of Formula I and pharmaceutically acceptable salts thereof, wherein all of the variables are as originally defined in the first class; and with the proviso that:

(A) when R² is unsubstituted phenyl or phenyl substituted with from 1 to 3 substituents each of which is independently halogen, NO₂, CN, C₁₋₄ alkyl, O—C₁₋₄ alkyl, SO₂NH₂, S(O)₂N(H)—C₁₋₄ alkyl, S(O)₂N(C₁₋₄ alkyl)₂, or C₁₋₄ haloalkyl, then R³ is not a 5-membered heteroaromatic ring containing from 1 to 3 heteroatoms selected from 1 to 3 N atoms, from zero to 1 O atom, and from zero to 1 S atom, wherein the heteroaromatic ring is connected to the rest of the molecule via a ring carbon, and the heteroaromatic ring is unsubstituted or substituted with from 1 to 2 substituents each of which is independently C₁₋₄ alkyl, Cl, Br, F, S(O)₂NH₂, S(O)₂N(H)—C₁₋₄ alkyl, S(O)₂N(C₁₋₄ alkyl)₂, CN, CycE, or C₁₋₄ haloalkyl.

A second sub-class of the first class includes compounds of Formula I and pharmaceutically acceptable salts thereof, wherein all of the variables are as originally defined in the first class; and with the proviso that:

(A) R² is not unsubstituted phenyl or substituted phenyl.

A third sub-class of the first class includes compounds of Formula I and pharmaceutically acceptable salts thereof, wherein all of the variables are as originally defined in the first class; proviso A as originally set forth in the first class is applied; and further provided that:

(B) R² is not phenyl that is di-substituted or tri-substituted with OCH₃.

A fourth sub-class of the first class includes compounds of Formula I and pharmaceutically acceptable salts thereof, wherein all of the variables are as originally defined in the first class; proviso A as originally set forth in the first class is applied; and further provided that:

(B) R² is not phenyl that is di-substituted or tri-substituted with O—C₁₋₄ alkyl.

A fifth sub-class of the first class includes compounds of Formula I and pharmaceutically acceptable salts thereof, wherein all of the variables are as originally defined in the first class; proviso A as set forth in the first sub-class of the first class is applied; and further provided that:

(B) R² is not phenyl that is di-substituted or tri-substituted with OCH₃.

A sixth sub-class of the first class includes compounds of Formula I and pharmaceutically acceptable salts thereof, wherein all of the variables are as originally defined in the first class; provso Aas set forth the first sub-class of the first class is applied; and further provide that:

(B) R² is not phenyl that is di-substituted or tri-substituted with O—C₁₋₄ alkyl.

A seventh sub-class of the first class includes compounds of Formula I and pharmaceutically acceptable salts thereof, wherein all of the variables are as originally defined in the first class; proviso A as originally set forth in the first class is applied; and further provided that:

(B) R² is not phenyl that is di-substituted or tri-substituted with OCH₃, and

(D) R³ is not an unsubstituted or substituted indazol-3-yl.

An eighth sub-class of the first class includes compounds of Formula I and pharmaceutically acceptable salts thereof, wherein all of the variables are as originally defined in the first class; proviso A as set forth in the first sub-class of the first class is applied; and provisos B and D as set forth in the seventh sub-class are applied.

A ninth sub-class of the first class includes compounds of Formula I and pharmaceutically acceptable salts thereof, wherein all of the variables are as originally defined in the first class; proviso A as set forth in the first sub-class of the first class is applied; and further provided that:

(B) R² is not phenyl that is di-substituted or tri-substituted with O—C₁₋₄ alkyl,

(D) R³ is not an unsubstituted or substituted indazol-3-yl.

A second class of the present invention includes compounds of Formula I and pharmaceutically acceptable salts thereof, wherein:
R³ is

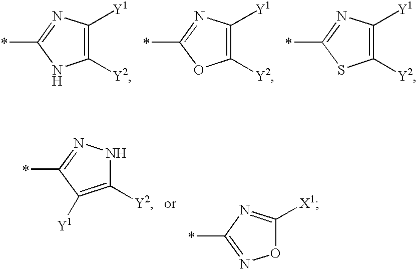

X¹ is:
   (1) H,
   (2) C₁₋₄ alkyl,
   (3) C₁₋₄ alkyl substituted with OH or O—C₁₋₄ alkyl,
   (4) C₁₋₄ alkyl substituted with from 2 to 4 OH,
   (5) C₃₋₆ cycloalkyl which is optionally substituted with C₁₋₄ alkyl or phenyl,
   (6) phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂,
   (7) phenyl substituted with a heterocyclic ring selected from the group consisting of:

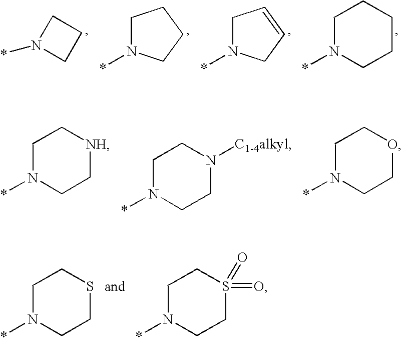

wherein the asterisk denotes the point of attachment to the rest of the molecule,
(8) CH₂-phenyl,
(9) CH₂—O-phenyl,
(10) heteroaryl selected from the group consisting of pyrrolyl, imidazolyl, furanyl, thienyl, oxazolyl, thiazolyl, pyridinyl, pyrimidinyl, and pyrazinyl, wherein the heteroaryl is optionally substituted with from 1 to 3 substituents each of which is independently Cl, Br, F, C₁₋₄ alkyl, CF₃, OH, O—C₁₋₄ alkyl, or OCF₃, or
(11) heteroaryl selected from the group consisting of 2,3-dihydrobenzo-1,4-dioxinyl and benzo-1,3-dioxolyl;
Y¹ independently has the same definition as X¹; and
Y² independently has the same definition as X¹;
or alternatively, Y¹ and Y² together with the carbon atoms to which each is attached form a benzo ring;
and all other variables are as originally defined in the first class; and with the proviso that:

(A) when R² is unsubstituted phenyl or phenyl substituted with from 1 to 3 substituents each of which is independently halogen, NO₂, CN, C₁₋₄ alkyl, O—C₁₋₄ alkyl, SO₂NH₂, or C₁₋₄ haloalkyl having from 1 to 3 halogen substituents, then X¹ in the definition of R³ is not H, C₁₋₄ alkyl, or C₃₋₅ cycloalkyl, and one of Y¹ and Y² in the definition of R³ is not H, C₁₋₄ alkyl, or C₃₋₅ cycloalkyl when the other of Y¹ and Y² is H, C₁₋₄ alkyl, or C₃₋₅ cycloalkyl.

A first sub-class of the second class includes compounds of Formula I and pharmaceutically acceptable salts thereof, wherein all of the variables are as originally defined in the second class; and with the proviso that:

(A) when R² is unsubstituted phenyl or phenyl substituted with from 1 to 3 substituents each of which is independently halogen, NO₂, CN, C₁₋₄ alkyl, O—C₁₋₄ alkyl, SO₂NH₂, S(O)₂N(H)—C₁₋₄ alkyl, S(O)₂N(C₁₋₄ alkyl)₂, or C₁₋₄ haloalkyl, then X¹ in the definition of R³ is not H, C₁₋₄ alkyl, or C₃₋₆ cycloalkyl which is optionally substituted with C₁₋₄ alkyl or phenyl, and one of Y¹ and Y² in the definition of R³ is (i) not H, C₁₋₄ alkyl, or C₃-₆ cycloalkyl which is optionally substituted with C₁₋₄ alkyl or phenyl when the other of Y¹ and Y² is H, C₁₋₄ alkyl, or C₃₋₆ cycloalkyl which is optionally substituted with C₁₋₄ alkyl or phenyl.

A second sub-class of the second class includes compounds of Formula I and pharmaceutically acceptable salts thereof, wherein all of the variables are as originally defined in the second class; and with the proviso that:

(A) R² is not unsubstituted phenyl or substituted phenyl.

A third sub-class of the second class includes compounds of Formula I and pharmaceutically acceptable salts thereof, wherein all of the variables are as originally defined in the second class; proviso A as originally set forth in the second class is applied; and further provided that:

(B) R² is not phenyl that is di-substituted or tri-substituted with OCH₃.

A fourth sub-class of the second class includes compounds of Formula I and pharmaceutically acceptable salts thereof, wherein all of the variables are as originally defined in the second class; proviso A as originally set forth in the second class is applied; and further provided that:

(B) R² is not phenyl that is di-substituted or tri-substituted with O—C₁₋₄ alkyl.

A fifth sub-class of the second class includes compounds of Formula I and pharmaceutically acceptable salts thereof, wherein all of the variables are as originally defined in the second class; proviso A as set forth in the first sub-class of the second class is applied; and further provided that:

(B) R² is not phenyl that is di-substituted or tri-substituted with OCH₃.

A sixth sub-class of the second class is identical to the fifth sub-class, except that proviso B is as follows: R² is not phenyl that is di-substituted or tri-substituted with O—C₁₋₄ alkyl.

A seventh sub-class of the second class includes compounds of Formula I and pharmaceutically acceptable salts thereof, wherein all of the variables are as originally defined in the second class; proviso A as originally set forth in the second class is applied; and further provided that:

(B) R² is not phenyl that is di-substituted or tri-substituted with OCH₃, and

(D) R³ is not an unsubstituted indazol-3-yl.

An eighth sub-class of the second class is identical to the seventh sub-class, except that proviso A as set forth in the first sub-class of the second class is applied.

A ninth sub-class of the second class is identical to the seventh sub-class, except that proviso B is as follows: R² is not phenyl that is di-substituted or tri-substituted with O—C₁₋₄ alkyl.

A third class of the present invention includes compounds of Formula I and pharmaceutically acceptable salts thereof, wherein:
R¹ is Cl or Br;
R² is:
   (i) phenyl, which is optionally substituted with a total of from 1 to 3 substituents, each of which is independently CH₃, OCH₃, CF₃, OCF₃, OH, Cl, Br, F, CN, C(O)N(CH₃)₂, C(O)CH₃, CO₂CH₃, or SO₂CH₃, or
   (ii) a saturated heterocyclic ring selected from the group consisting of:

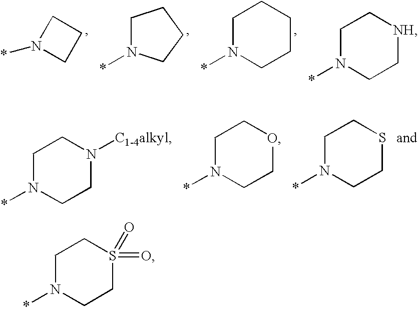

wherein the asterisk denotes the point of attachment to the rest of the molecule,
R³ is

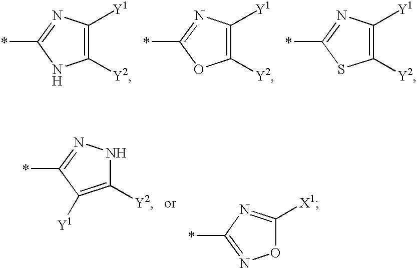

X¹ is:
   (1) H,
   (2) C₁₋₃ alkyl,
   (3) C₁₋₃ alkyl substituted with OH or OCH₃,
   (4) C₁₋₄ alkyl substituted with from 2 to 4 OH,
   (5) C₃₋₆ cycloalkyl which is optionally substituted with C₁₋₄ alkyl or phenyl,
   (6) phenyl which is optionally substituted with from 1 to 3 substituents each of which is independently CH₃, OCH₃, CF₃, OCF₃, OH, Cl, Br, F, CN, NO₂, C(O)N(H)CH₃, C(O)N(CH₃)₂, CO₂CH₃, or S(O)₂CH₃,
   (7) phenyl substituted with a saturated heterocyclic ring selected from the group consisting of:

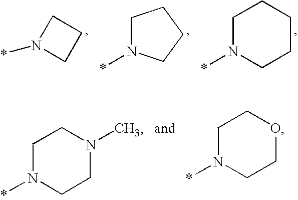

wherein the asterisk denotes the point of attachment to the rest of the molecule,
(8) CH₂-phenyl,
(9) CH₂—O-phenyl,
(10) thienyl or pyridinyl, or
(11) benzo-1,3-dioxolyl;
one of Y¹ and Y² independently has the same definition as X¹, and the other of Y¹ and Y² is H; or alternatively, Y¹ and Y² together with the carbon atoms to which each is attached form a benzo ring;
R⁴ is H; and
R⁵ is H;
and with the proviso that:

(A) when R² is unsubstituted phenyl or phenyl substituted with from 1 to 3 substituents each of which is independently CH₃, OCH₃, CF₃, Cl, Br, F, or CN, then (i) X¹ in the definition of R³ is not H, C₁₋₃ alkyl, or C₃₋₅ cycloalkyl and (ii) one of Y¹ and Y² in the definition of R³ is not H, C₁₋₄ alkyl, or C₃₋₅ cycloalkyl when the other of Y¹ and Y² is H.

A first sub-class of the third class includes compounds of Formula I and pharmaceutically acceptable salts thereof, wherein all of the variables are as originally defined in the third class; and with the proviso that:

(A) when R² is unsubstituted phenyl or phenyl substituted with from 1 to 3 substituents each of which is independently CH₃, OCH₃, CF₃, Cl, Br, F, or CN, then (i) X¹ in the definition of R³ is not H, C₁₋₃ alkyl, or C₃₋₆ cycloalkyl which is optionally substituted with C₁₋₄ alkyl or phenyl,and (ii) one of Y¹ and Y² in the definition of R³ is not H, C₁₋₃ alkyl, or C₃₋₆ cycloalkyl which is optionally substituted with C₁₋₄ alkyl or phenyl when the other of Y¹ and Y² is H.

A second sub-class of the third class includes compounds of Formula I and pharmaceutically acceptable salts thereof, wherein all of the variables are as originally defined in the third class; and with the proviso that:

(A) R² is not unsubstituted phenyl or substituted phenyl.

A third sub-class of the third class includes compounds of Formula I and pharmaceutically acceptable salts thereof, wherein all of the variables are as originally defined in the third class; proviso A as originally set forth in the third class is applied; and further provided that:

(B) R² is not phenyl that is di-substituted or tri-substituted with OCH₃.

A fourth sub-class of the third class includes compounds of Formula I and pharmaceutically acceptable salts thereof, wherein all of the variables are as originally defined in the second class; proviso A as set forth in the first sub-class of the third class is applied; and further provided that:

(B) R² is not phenyl that is di-substituted or tri-substituted with OCH₃.

A fifth sub-class of the third class includes compounds of Formula I and pharmaceutically acceptable salts thereof, wherein all of the variables are as originally defined in the third class; proviso A as originally set forth in the third class is applied; and further provided that:

(B) R² is not phenyl that is di-substituted or tri-substituted with OCH₃, and

(D) R³ is not an unsubstituted indazol-3-yl.

A sixth sub-class of the third class is identical to the fifth sub-class, except that proviso A as set forth in the first sub-class of the third class is applied.

A fourth class of the present invention includes compounds of Formula I and pharmaceutically acceptable salts thereof, wherein:
R¹ is Cl or Br;
R² is phenyl and R³ is

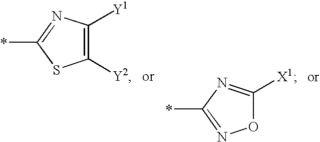

R² is

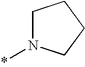

### and R³ is

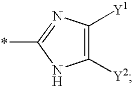 [0540] X¹, Y¹ and Y² are each as defined in the third class;
R⁴ is H; and
R⁵ is H;
and with the proviso that:

(A) when R² is unsubstituted phenyl, then X¹ in the definition of R³ is not H, C₁₋₃ alkyl, or C₃₋₅ cycloalkyl, and one of Y¹ and Y² in the definition of R³ is (i) not H, C₁₋₃ alkyl, or C₃₋₅ cycloalkyl when the other of Y¹ and Y² is H.

A first sub-class of the fourth class includes compounds of Formula I and pharmaceutically acceptable salts thereof, wherein all of the variables are as originally defined in the fourth class; and with the proviso that:

(A) when R² is unsubstituted phenyl, then X¹ in the definition of R³ is not H, C₁₋₃ alkyl, or C₃₋₆ cycloalkyl which is optionally substituted with C₁₋₄ alkyl or phenyl, and one of Y¹ and Y² in the definition of R³ is (i) not H, C₁₋₃ alkyl, or C₃₋₆ cycloalkyl which is optionally substituted with C₁₋₄ alkyl or phenyl when the other of Y¹ and Y² is H.

A second sub-class of the fourth class includes compounds of Formula I and pharmaceutically acceptable salts thereof, wherein all of the variables are as originally defined in the fourth class; and with the proviso that:

(A) R² is not unsubstituted phenyl.

A third sub-class of the fourth class includes compounds of Formula I and pharmaceutically acceptable salts thereof, wherein all of the variables are as originally defined in the fourth class; proviso A as originally set forth in the fourth class is applied; and further provided that:

(D) R³ is not an unsubstituted indazol-3-yl.

A fourth sub-class of the fourth class is identical to the third sub-class, except that proviso A as set forth in the first sub-class of the fourth class is applied.

A fifth sub-class of the fourth class is identical to the third sub-class, except that proviso A as set forth in the second sub-class of the fourth class is applied.

A fifth class of the present invention includes compounds of Formula I and pharmaceutically acceptable salts thereof, wherein:
R² is:
   (1) C₁₋₆ alkyl,
   (2) CycB, or
   (3) C₁₋₆ alkyl substituted with CycB;
CycB is as originally defined;
and all other variables are as originally defined in the first class of the present invention.

A sixth class of the present invention includes compounds of Formula I and pharmaceutically acceptable salts thereof, wherein:
R¹ is halogen;
R² is:
   (1) C₁₋₆ alkyl,
   (2) C₃₋₆ cycloalkyl, or
   (3) C₁₋₆ alkyl substituted with C₃₋₆ cycloalkyl;
R³ is

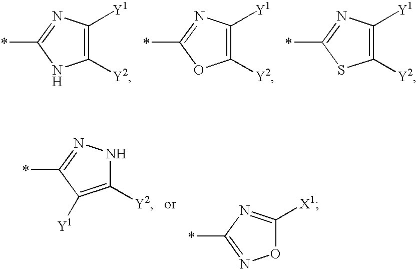

X¹ is:
   (1) H,
   (2) C₁₋₄ alkyl,
   (3) C₁₋₄ alkyl substituted with OH or O—C₁₋₄ alkyl,
   (4) C₁₋₄ alkyl substituted with from 2 to 4 OH,
   (5) C₃₋₆ cycloalkyl which is optionally substituted with C₁₋₄ alkyl or phenyl,
   (6) phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂,
   (7) vhenvl substituted with a heterocyclic ring selected from the group consisting of:

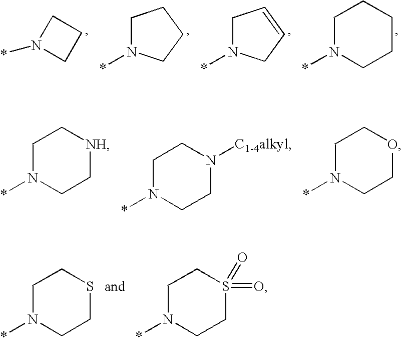

wherein the asterisk denotes the point of attachment to the rest of the molecule,
(8) CH₂-phenyl,
(9) CH₂—O-phenyl,
(10) heteroaryl selected from the group consisting of pyrrolyl, imidazolyl, furanyl, thienyl, oxazolyl, thiazolyl, pyridinyl, pyrimidinyl, and pyrazinyl, wherein the heteroaryl is optionally substituted with from 1 to 3 substituents each of which is independently Cl, Br, F, C₁₋₄ alkyl, CF₃, OH, O—C₁₋₄ alkyl, or OCF₃, or
(11) heteroaryl selected from the group consisting of 2,3-dihydrobenzo-1,4-dioxinyl and benzo-1,3-dioxolyl;
Y¹ independently has the same definition as X¹; and
Y² independently has the same definition as X¹;
or alternatively, Y¹ and Y² together with the carbon atoms to which each is attached form a benzo ring;
R⁴ is H; and
R⁵ is H.

A seventh class of the present invention includes compounds of Formula I and pharmaceutically acceptable salts thereof, wherein:
R¹ is Cl or Br;
R² is:
   (1) C₁₋₅ alkyl,
   (2) C₃₋₆ cycloalkyl, or
   (3) (CH₂)₁₋₂—C₃₋₆ cycloalkyl;
R³ is

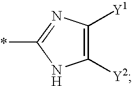

one of Y¹ and Y² is H, and the other of Y¹ and Y² is:
   (1) H,
   (2) C₁₋₃ alkyl,
   (3) C₁₋₃ alkyl substituted with OH or OCH₃,
   (4) C₁₋₄ alkyl substituted with from 2 to 4 OH,
   (5) C₃₋₆ cycloalkyl which is optionally substituted with C₁₋₄ alkyl or phenyl,
   (6) phenyl which is optionally substituted with from 1 to 3 substituents each of which is independently CH₃, OCH₃, CF₃, OCF₃, OH, Cl, Br, F, CN, NO₂, C(O)N(H)CH₃, C(O)N(CH₃)₂, CO₂CH₃, or S(O)₂CH₃,
   (7) phenyl substituted with a saturated heterocyclic ring selected from the group consisting of

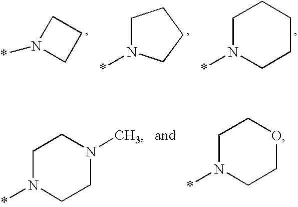

wherein the asterisk denotes the point of attachment to the rest of the molecule,
(8) CH₂-phenyl,
(9) CH₂—O-phenyl,
(10) thienyl or pyridinyl, or
(11) benzo-1,3-dioxolyl;
R⁴ is H; and
R⁵ is H.

Another embodiment of the present invention is a compound, or a pharmaceutically acceptable salt thereof, selected from the group consisting of the compounds set forth in Examples 1 to 37 below. In an aspect of this embodiment, the compound is selected from the group consisting of the compounds set forth in Examples 1 to 15. In another aspect of this embodiment, the compound is selected from the group consisting of the compounds set forth in Examples 16 to 33. In still another aspect, the compound is selected from the group consisting of the compounds set forth in Examples 34 to 37.

Another embodiment of the present invention is a compound of Formula I, or a pharmaceutically acceptable salt thereof, as originally defined or as defined in any of the foregoing embodiments, classes, sub-classes, aspects, or features, wherein the compound or its salt is substantially pure. As used herein “substantially pure” means that the compound or its salt is present (e.g., in a product isolated from a chemical reaction or a metabolic process) in an amount of at least about 90 wt. % (e.g., from about 95 wt. % to 100 wt. %), preferably at least about 95 wt. % (e.g., from about 98 wt. % to 100 wt. %), more preferably at least about 99 wt. %, and most preferably 100 wt. %. The level of purity of the compounds and salts can be determined using standard methods of analysis. A compound or salt of 100% purity can alternatively be described as one which is free of detectable impurities as determined by one or more standard methods of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

Other embodiments of the present invention include the following:

(a) A pharmaceutical composition comprising an effective amount of Compound I as originally defined above (including proviso A), or a pharmaceutically acceptable salt thereof, and a pharmaceutically acceptable carrier.

(b) A pharmaceutical composition which comprises the product prepared by combining (e.g., mixing) an effective amount of Compound I as originally defined above (including proviso A), or a pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV reverse transcriptase inhibitors other than a compound of Formula I, and HIV integrase inhibitors.

(e) A pharmaceutical combination which is (i) a compound of Formula I as originally defined above (including proviso A), or a pharmaceutically acceptable salt thereof, and (ii) an anti-HIV agent selected from the group consisting of HIV antiviral agents, immunomodulators, and anti-infective agents; wherein the compound of Formula I and the anti-HIV agent are each employed in an amount that renders the combination effective for inhibition of HIV reverse transcriptase, for treatment or prophylaxis of infection by HIV, or for treatment, prophylaxis of, or delay in the onset of AIDS.

(f) The combination of (e), wherein the anti-HIV agent is an antiviral selected from the group consisting of HIV protease inhibitors, HIV reverse transcriptase inhibitors other than a compound of Formula I, and HIV integrase inhibitors.

Additional embodiments of the invention include the pharmaceutical compositions and combinations set forth in (a)-(f) above, wherein the compound of the present invention employed therein is a compound defined in one of the embodiments, classes, or sub-classes described above, wherein it is understood that the definitions include the accompanying provisos. In all of these embodiments, the compound can optionally be used in the form of a pharmaceutically acceptable salt.

Additional embodiments of the present invention include each of the pharmaceutical compositions and combinations set forth in (a)-(f) above and embodiments thereof, wherein the compound of the present invention or its salt employed therein is substantially pure. With respect to a pharmaceutical composition comprising a compound of Formula I or its salt and a pharmaceutically acceptable carrier and optionally one or more excipients, it is understood that the term “substantially pure” is in reference to Compound I or its salt per se; i.e., the purity of the active ingredient in the composition.

The present invention also includes a method for inhibition of HIV reverse transcriptase, for treatment or prophylaxis of HIV infection, or for treatment, prophylaxis of, or delay in the onset of AIDS, which comprises administering, to a subject in need thereof an effective amount of a compound of Formula I, or a pharmaceutically acceptable salt thereof, wherein Formula I is as originally set forth and defined above (including proviso A). Embodiments of the method of the present invention include those in which the compound of Formula I administered to the subject is as defined in the compound embodiments, classes and sub-classes set forth above, except that any of provisos B to G included therein are not applied. In sub-embodiments of each of these method embodiments, the provisos B to G are applied to the extent they are included in the corresponding compound embodiment, class or sub-class.

The present invention also includes a compound of Formula I, or a pharmaceutically acceptable salt thereof, (i) for use in, (ii) for use as a medicament for, or (iii) for use in the preparation of a medicament for: (a) inhibition of HIV reverse transcriptase, (b) treatment or prophylaxis of infection by HIV, or (c) treatment, prophylaxis of, or delay in the onset of AIDS. In these uses, the compound of Formula I is as originally set forth and defined above, including proviso A (i.e., proviso A is applied). In these uses, the compounds of the present invention can optionally be employed in combination with one or more anti-HIV agents selected from HIV antiviral agents, anti-infective agents, and immunomodulators. Embodiments of the uses of the present invention include those in which the compound of Formula I is as defined in the compound embodiments, classes and sub-classes set forth above, except that any of provisos B to G included therein are not applied. In sub-embodiments of these use embodiments, the provisos B to G are included in the definition of the compound to the extent they are included in the corresponding compound embodiment, class or sub-class.

As used herein, the term “alkyl” refers to any linear or branched chain alkyl group having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, and isopropyl, ethyl and methyl. As another example, “C₁₋₄ alkyl” refers to n-, iso-, sec- and t-butyl, n- and isopropyl, ethyl and methyl.

The term “alkylene” refers to any divalent linear or branched chain aliphatic hydrocarbon radical (or alternatively an “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest an alkylene selected from the group consisting of —CH₂—, —CH(CH₃)—, and —C(CH₃)₂—.

The term “cycloalkyl” refers to any cyclic ring of an alkane having a number of carbon atoms in the specified range. Thus, for example, “C₃₋₈ cycloalkyl” (or “C₃-C₈ cycloalkyl”) refers to cyclopropyl, cyclobutyl, cyclopentyl, cyclohexyl, cycloheptyl, and cyclooctyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” appearing in the definition of a functional group (e.g., “C(O)R^{A}”) refers to carbonyl. The term “S(O)₂” or “SO₂” appearing in the definition of a functional group refers to sulfonyl, the term “S(O)” refers to sulfinyl, and the terms “C(O)O” and “CO₂” both refer to carboxyl

The left-most atom or variable shown in any of the groups in the definitions of R¹ to R⁵ is the atom or variable attached to or nearest to the indole ring. Thus, for example, a compound of the present invention in which R¹ is J-AryA, J in the definition of R¹ is C(O)N(R^{A}), R⁴ is L-CyC, and L is C(O)CH₂, R⁵═H, and R²=phenyl, is as follows:

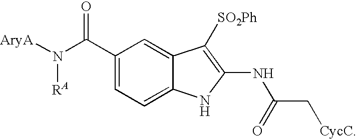

The symbols “*” and “ [symbol] ” at the end of a bond each refer to the point of attachment of a functional group or other chemical moiety to the rest of the molecule of which it is a part.

Unless expressly stated to the contrary in a particular context, any of the various carbocyclic and heterocyclic rings and ring systems defined herein may be attached to the rest of the compound at any ring atom (i.e., any carbon atom or any heteroatom) provided that a stable compound results. Suitable aryls include phenyl, 9- and 10-membered bicyclic, fused carbocyclic ring systems, and 11- to 14-membered tricyclic fused carbocyclic ring systems, wherein in the fused carbocyclic ring systems at least one ring is aromatic. Suitable aryls include, for example, phenyl, naphthyl, tetrahydronaphthyl (tetralinyl), indenyl, anthracenyl, and fluorenyl. Suitable heteroaryls include 5- and 6-membered heteroaromatic rings and 9- and 10-membered bicyclic, fused ring systems in which at least one ring is aromatic, wherein the heteroaromatic ring or the bicyclic, fused ring system contains from 1 to 4 heteroatoms independently selected from N, O and S, wherein each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, thazolyl, tetrazolyl, oxazolyl, isooxazolyl, oxadiazolyl, oxatriazolyl, thiazolyl, isothiazolyl, and thiadiazolyl. Suitable heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl (e.g., benzo-1,3-dioxolyl:

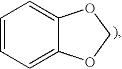

benzopiperidinyl, benzisoxazolyl, benzoxazolyl, chromanyl, isochromanyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl

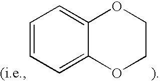

Suitable saturated and mono-unsaturated heteroCyclic rings include 4- to 7-membered saturated and mono-unsaturated heterocyclic rings containing at least one carbon atom and from 1 to 4 heteroatoms independently selected from N, O and S, wherein each S is optionally oxidized to S(O) or S(O)₂. Suitable 4- to 7-membered saturated heterocyclics include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Suitable mono-unsaturated heterocyclic rings include those corresponding to the saturated heterocyclic rings listed in the preceding sentence in which a single bond is replaced with a double bond (e.g., a carbon-carbon single bond is replaced with a carbon-carbon double bond). Suitable saturated and mono-unsaturated heterobicyclic rings include 6- to 10-membered saturated and mono-unsaturated, bridged or fused heterobicyclic rings containing from 1 to 4 heteroatoms independently selected from N, O and S, where each S is optionally oxidized to S(O) or S(O)₂, Suitable saturated heterobicyclics include:

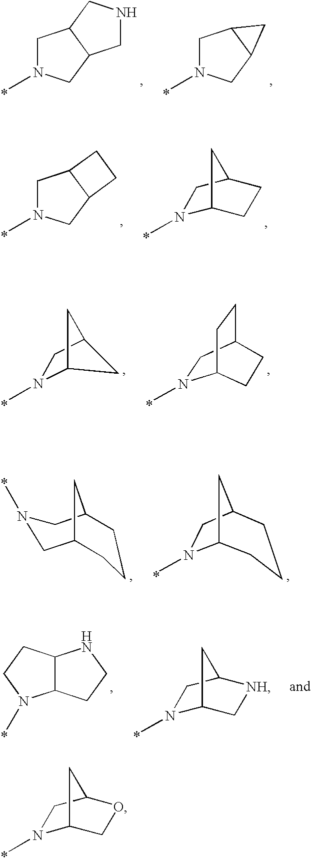

and suitable mono-unsaturated heterobicyclics include those corresponding to the foregoing saturated heterobicyclics in which a single bond is replaced with a double bond. It is understood that the specific rings and ring systems suitable for use in the present invention are not limited to those listed in this paragraph. The rings and ring systems listed in this paragraph are merely representative.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. As another example, an aryl or heteroaryl described as optionally substituted with “from 1 to 5 substituents” is intended to include as aspects thereof, an aryl or heteroaryl optionally substituted with 1 to 4 substituents, 1 to 3 substituents, 1 to 2 substituents, 2 to 5 substituents, 2 to 4 substituents, 2 to 3 substituents, 3 to 5 substituents, 3 to 4 substituents, 4 to 5 substituents, 1 substituent, 2 substituents, 3 substituents, 4 substituents, and 5 substituents.

When any variable (e.g., R^{A}, R^{B}, AryE, or HetE) occurs more than one time in⁻any constituent or in Formula I or in any other formula depicting and describing compounds employed in the invention, its definition on each occurrence is independent of its definition at every other occurrence. Also, combinations of substituents and/or variables are permissible only if such combinations result in stable compounds.

The term “substituted” (e.g., as in “is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., cycloalkyl, aryl, or heteroaryl) provided such ring substitution is chemically allowed and results in a stable compound. Ring substituents can be attached to the ring atom which is attached to the rest of the molecule; e.g., methyl-substituted 3-oxetanyl refers to:

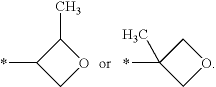

As a result of the selection of substituents and substituent patterns, certain compounds of the present invention can exhibit keto-enol tautomerism. All tautomeric forms of these compounds, whether individually or in mixtures, are within the scope of the present invention. For example, in instances where a hydroxy (—OH) substituent(s) is (are) permitted on a heteroaromatic ring and keto-enol tautomerism is possible, it is understood that the substituent might in fact be present, in whole or in part, in the keto form, as exemplified here for a hydroxypyridinyl substituent:

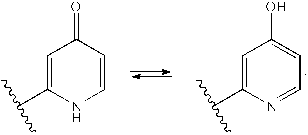

Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

The method of the present invention involves the use of compounds of the present invention in the inhibition of HIV reverse transcriptase (wild type and/or mutant strains thereof), the prophylaxis or treatment of infection by human immunodeficiency virus (HIV) and the prophylaxis, treatment or delay in the onset of consequent pathological conditions such as AIDS. Prophylaxis of AIDS, treating AIDS, delaying the onset of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an HIV-infected female who is nursing (i.e., breast feeding) a child to the child via administration of an effective amount of a compound of Formula I, or a pharmaceutically acceptable salt thereof.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Certain of the compounds employed in the present invention carry an acidic moiety (e.g., —COOH or a phenolic group), in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of Formula I mean providing the compound or a prodrug of the compound to the individual in need of treatment or prophylaxis. When a compound or a prodrug thereof is provided in combination with one or more other active agents (e.g., antiviral agents useful for treating or prophylaxis of HIV infection or AIDS), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV reverse transcriptase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

In the method of the present invention (i.e., inhibiting HIV reverse transcriptase, treating or prophylaxis of HIV infection or treating, prophylaxis of, or delaying the onset of AIDS), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrastemal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and, of ingredients suitable for use in said compositions is provided in Remington's Pharmaceutical Sciences, 18^{th} edition, edited by A. R. Gennaro, Mack Publishing Co., 1990.

The compounds of Formula I can be administered orally in a dosage range of 0.001 to 1000 mg/kg of mammal (e g , human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to the use of the compounds of Formula I in combination with one or more agents useful in the treatment of HIV infection or AIDS. For example, the compounds of Formula I can be effectively administered, whether at periods of pre-exposure and/or post-exposure, in combination with effective amounts of one or more HIV antiviral agents, imunomodulators, antiinfectives, or vaccines useful for treating HIV infection or AIDS, such as those disclosed in Table 1 of WO 01/38332 or in the Table in WO 02/30930. Suitable HIV antiviral agents for use in combination with the compounds of Formula I include, for example, HIV protease inhibitors (e.g., indinavir, atazanavir, lopinavir optionally with ritonavir, saquinavir, or nelfinavir), nucleoside HIV reverse transcriptase inhibitors (e.g., abacavir, lamivudine (3TC), zidovudine (AZT), or tenofovir), non-nucleoside HIV reverse transcriptase inhibitors (e.g., efavirenz or nevirapine), and HIV integrase inhibitors such as those described in WO 02/30930, WO 03/35076, and WO 03/35077. It will be understood that the scope of combinations of compounds of Formula I with HIV antiviral agents, immunomodulators, anti-infectives or vaccines is not limited to the foreogoing substances or to the list in the above-referenced Tables in WO 01/38332 and WO 02/30930, but includes in principle any combination with any pharmaceutical composition useful for the treatment of HIV infection or AIDS. The HIV antiviral agents and other agents will typically be employed in these combinations in their conventional dosage ranges and regimens as reported in the art, including, for example, the dosages described in the Physicians' Desk Reference, 58^{th} edition, Thomson PDR, 2004. The dosage ranges for a compound of Formula I in these combinations are the same as those set forth above. It is understood that pharmaceutically acceptable salts of the compounds of the invention and/or the other agents (e.g., indinavir sulfate) can be used as well.

Abbreviations employed herein include the following:
DCM=dichloromethane
dGTP=deoxyguanosine triphosphate
DME=dimethoxyethane
DMSO=dimethylsufoxide
dNTP=deoxynucleoside triphosphate
EDTA=ethylenediaminetetracetic acid
EGTA=ethylene glycol bis(2-aminoethyl ether)-N,N,N′,N′-tetraacetic acid
ES MS=electrospray mass spectroscopy
Et=ethyl
HRMS=high resolution mass spectroscopy
LAH=lithium aluminum hydride
LC=liquid chromatography
MeOH=methanol
MS=mass spectroscopy
NMR=nuclear magnetic resonance
Ph=phenyl

1TEA=triethylamine
TFA=trifluoroacetic acid
TFAA=trifluoroacetic anhydride
THF=tetrahydrofuran

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Scheme 1 provides a method for preparing 2-thiazolylindoles and 2-oxadiazolylindoles, wherein indole-2-carboxamide 1 (see Williams, T. M., et al., J. Med. Chem. 1993, 36, 1291) is reacted with TFAA under basic conditions (e.g., in the presence of a base such a pyridine) to furnish nitrile 2, which can be reacted with hydroxylamine or an acid salt thereof (e.g., HCl) to afford hydroxyamidine 3, for example, by refluxing the nitrile 2 and NH₂OH overnight in a suitable solvent (e.g., an alcohol such as EtOH) and in the presence of a base (e.g., a trialkyl amine such as triethylamine). Acylation of 3 with a suitable acid halide (e.g., using an acid chloride in a suitable solvent—e.g., an ether such as DME—and in the presence of a base such as pyridine) and cyclization at an elevated temperature (e.g., in a microwave reactor) affords furnishes the desired oxadiazole 4. Alternatively, nitrile 2 can be treated with ammonium sulfide to obtain thioamide 5, which can be heated (e.g., via microwaves) with a substituted α-bromoketone in a suitable solvent (e.g., acetone) to provide the final thiazole 6.

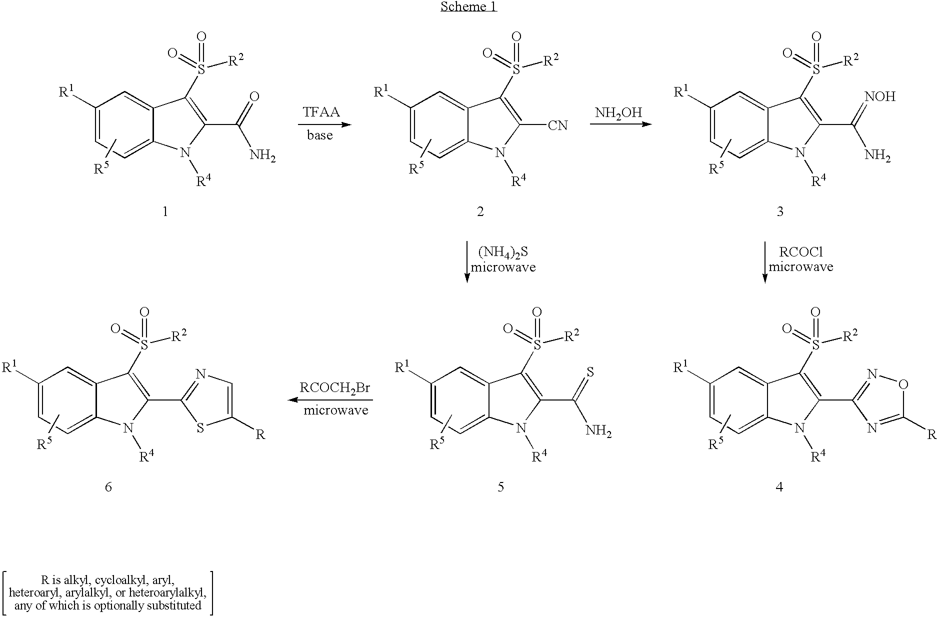

Scheme 2 provides a method for the preparation of 2-imidazol-2-ylindoles, wherein indole-2-carboxylic ester 7 (see Young et al., Bioorg. Med. Chem. Lett. 1995, 5, p. 491) is reduced with a suitable reducing agent (e.g., LAH in THF at low temperature—e.g., 0-5° C.) to furnish alcohol 8 which can be immediately oxidized to aldehyde 9 with the Dess-Martin periodinate. Condensation with a suitable a-ketoaldehyde at elevated temperature (e.g., 60-100° C. in a microwave reactor) provides the final imidazole 10.

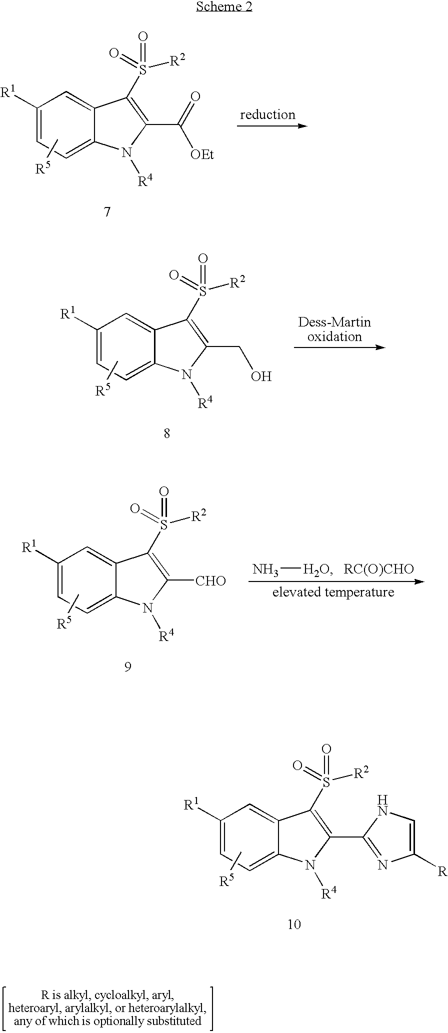

In the processes for preparing compounds of the present invention set forth in the foregoing schemes, functional groups in various moieties and substituents may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{th} edition, 1999, and 2^{nd} edition, 1991. The protecting groups may be removed at a convenient subsequent stage using methods known in the art. Alternatively the interfering group can be introduced into the molecule subsequent to the reaction step of concern.

The following examples serve only to illustrate the invention and its practice. The examples are not to be construed as limitations on the scope or spirit of the invention.

The compounds set forth in Examples 1-33 were purified by LCMS and the purified product obtained as a TFA salt. The LCMS conditions employed to purify the product and obtain the salt were as follows: column: 20 cm×50 mm Phenomenex GEMINI C18 column; mobile phase: A=0.1% TFA in water, B=CH₃CN, 0 minutes (15% B, 15 mL/min), 0.8 minutes (15% B, 25 mL/min), 8.3 minutes (40% B, 25 mL/min), 8.4 minutes (100% B, 25 mL/min); wavelength: 214 nm; column temperature: ambient.

### Example 1

### 5-chloro-2-[5-(methoxymethyl)-1,2,4-oxadiazol-3-yl]-3-(phenylsulfonyl)-1H-indole

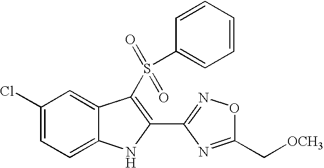

### Step 1: 5-Chloro-3-(phenylsulfonyl)-1H-indole-2-carbonitrile

TFAA (4.23 g, 15 mmol) was added to a stiffed solution of 5-chloro-3-(phenylsulfonyl)-1H-indole-2-carboxamide (1.01 g, 3.0 mmol) in pyridine/DCM (1:1, 40 mL) at 0° C. (with an ice bath). After addition, the ice bath was removed and the resulting mixture was stirred at room temperature for 4 hours. after which 2M NH₃-MeOH solution was added to the reaction mixture and the admixture was heated at 40° C. overnight. The reaction was then concentrated and the residue treated with DCM/water (300 mL:100 mL). The DCM solution was separated and washed with water, 2N HCl, brine dried over Na₂SO₄, filtered, and concentrated. The concentrated residue was purified by LCMS to afford the desired 5-chloro-3-(phenylsulfonyl)-1H-indole-2-carbonitrile as a white solid. Analytical LCMS: single peak (214 nm), 3.199 min, ES MS (M+1)=317.

### Step 2: 5-Chloro-N′-hydroxy-3-(phenylsulfonyl)-1H-indole-2-carboximidamide

A mixture of 5-chloro-3-(phenylsulfonyl)-1H-indole-2-carbonitrile (63 mg, 0.20 mmol), NH₂OH (HCl salt, 150 mg, 2.0 mmol), and TEA (260 mg, 2.0 mmol) in EtOH (3 mL) was refluxed overnight. The reaction mixture was then concentrated and treated with water/DCM (20 mL:60 mL). The DCM solution was separated and washed with brine, dried over Na₂SO₄, filtered, concentrated to afford the desired product 5-chloro-N′-hydroxy-3-(phenylsulfonyl)-1H-indole-2-carboximidamide as a white solid. Analytical LCMS: single peak (214 nm), 2.769 min, ES MS (M+1)=350.

### Step 3: 5-Chloro-2-[5-(methoxymethyl)-1,2,4-oxadiazol-3-yl]-3-(phenylsulfonyl)-1H-indole

A mixture of 5-chloro-N-hydroxy-3-(phenylsulfonyl)-1H-indole-2-carboximidamide (35 mg, 0.10 mmol), MeOCH₂COCl (16 mg, 0.15 mmol), and pyridine (200 pL) in DME (2 mL) was heated at 160° C. in a microwave for 10 minutes. The reaction mixture was then cooled down and concentrated, and the residue purified by LCMS to give the pure product 5-chloro-2-[5-(methoxymethyl)-1,2,4-oxadiazol-3-yl]-3-(phenylsulfonyl)-1H-indole as a TFA salt (yellow solid). Analytical LCMS: single peak (214 nm), 3.216 min, ES MS (M+1)=404.8; ¹H NMR (500 MHz, d₆-DMSO) δ 13.49 (s, 1H), 8.20-8.13 (m, 3H), 7.69-7.59 (m, 4H), 7.44 (dd, J=8.9, 2.0 Hz, 1H), 4.95 (s, 2H), 3.48 (s, 3H); HRMS, calc'd for C₁₈H₁₅ClN₃O₄S. (M+H), 404.0467; found 404.0461.

### Example 2

### 5-chloro-3-(phenylsulfonyl)-2-(5-pyridin-4-yl-1,3-thiazol-2-yl)-1H-indole

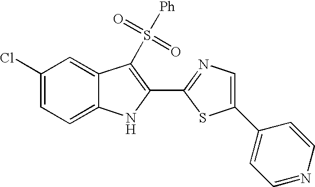

### Step 1: 5-Chloro-3-(phenylsulfonyl)-1H-indole-2-carbothioamide

A mixture of 5-chloro-3-(phenylsulfonyl)-1H-indole-2-carbonitrile (96 mg, 0.3 mmol; see Step 1 of Example 1), (NH4)2S (50% w/w water solution, excess) and TEA (0.5 mL, excess) in pyridine (2 mL) was microwaved at 120° C. for 30 minutes, after which the mixture was cooled down and concentrated, and the residue was then purified by LCMS to provide the title compound as a yellow solid. Analytical LCMS: single peak (214 nm), 3.224 min, ES MS (M+1)=351.

### Step 2: 5-Chloro-3-(phenylsulfonyl)-2-(5-pyridin-4-yl-1,3-thiazol-2-yl)-1H-indole

A mixture of 5-chloro-3-(phenylsulfonyl)-1H-indole-2-carbothioamide (35 mg, 0.1 mmol) and 2-bromo-1-pyridin-4-ylethanone (HBr salt, 31 mg, 0.11 mmol)) in acetone was microwaved at 100° C. for 20 minutes, after which the mixture was cooled down and concentrated and the residue purified by LCMS to provide the title compound as a TFA salt (brown solid). Analytical LCMS: single peak (214 nm), 2.709 min, ES MS (M+1)=452; ¹H NMR (500 MHz, d₆-DMSO) δ 13.23 (s, 1H), 9.4 (s, 1H), 8.90 (d, J=5.6, Hz, 2H), 8.38 (d, J=5.7, Hz, 2H), 8.10 (d, J=1.9, Hz, 1 H), 8.00 (d, J=7.5, Hz, 2H), 7.69-7.57 (m, 4H), 7.45 (dd, J=8.8, 2.0 Hz, 1H); HRMS, calc'd for C₂₂H₁₅ClN₃O₂S₂ (M+H), 452.0289; found 452.0284.

### Examples 3-15

The compounds in the following table were prepared in accordance with the procedures set forth in Example 1 and Example 2. All of the compounds in the table were prepared as TFA salts. The compound name shown in the table is the name of the free base.

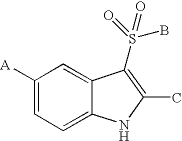

| | | | | | ES MS |
|---|---|---|---|---|---|
| Example | Name | A | B | C | (M + 1) |
| 3 | 5-chloro-3-(phenylsulfonyl)-2- (4-pyridin-2-yl-1,3-thiazol-2-yl)- 1H-indole | Cl | Ph | 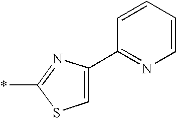 | 453.0 |
| 4 | 5-chloro-3-(phenylsulfonyl)-2- (4-pyridin-3-yl-1,3-thiazol-2-yl)- 1H-indole | Cl | Ph | 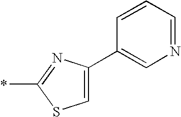 | 453.0 |
| 5 | 5-chloro-2-[5-(2-chlorophenyl)- l,2,4-oxadiazol-3-yl]-3- (phenylsulfonyl)-1H-indole | Cl | Ph | 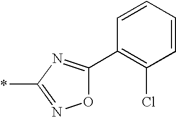 | 471.3 |
| 6 | 5-chloro-3-(phenylsulfonyl)-2- (5-propyl-l,2,4-oxadiazol-3-yl)- 1H-indole | Cl | Ph | 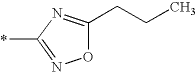 | 402.9 |
| 7 | 5-chloro-2-[5-(2-fluorophenyl)- 1,2,4-oxadiazol-3-yl]-3- (phenylsulfonyl)-1H-indole | Cl | Ph | 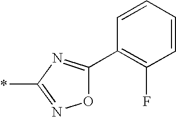 | 454.9 |
| 8 | 5-chloro-2-{5-[(1R,2R)-2- phenylcyclopropyl]-1,2,4- oxadiazol-3-yl}-3- (phenylsulfonyl)-1H-indole | Cl | Ph | 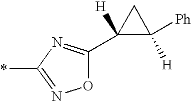 | 477.0 |
| 9 | 5-chloro-2-[5-(phenoxymethyl)- 1,2,4-oxadiazol-3-yl]-3- (phenylsulfonyl)-1H-indole | Cl | Ph | 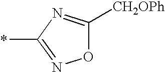 | 466.9 |
| 10 | 5-chloro-3-(phenylsulfonyl)-2- (5-pyridin-4-yl-1,2,4-oxadiazol- 3-yl)-1H-indole | Cl | Ph | 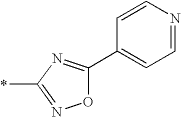 | 437.9 |
| 11 | 5-chloro-2-[5-(2,4- difluorophenyl)-1,2,4-oxadiazol- 3-yl]-3-(phenylsulfonyl)-1H- indole | Cl | Ph | 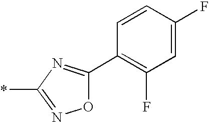 | 472.9 |
| 12 | 5-chloro-2-(5-methyl-1,2,4- oxadiazol-3-yl)-3- (phenylsulfonyl)-1H-indole | Cl | Ph | 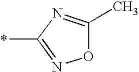 | 374.8 |
| 13 | 5-chloro-2-(5-cyclobutyl-1,2,4- oxadiazol-3-yl)-3- (phenylsulfonyl)-1H-indole | Cl | Ph | 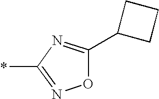 | 414.9 |
| 14 | 2-(5-benzyl-1,2,4-oxadiazol-3- yl)-5-chloro-3-(phenylsulfonyl)- 1H-indole | Cl | Ph | 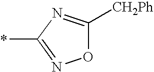 | 450.9 |
| 15 | 5-chloro-2-(5-ethyl-1,2,4- oxadiazol-3-yl)-3- (phenylsulfonyl)-1H-indole | Cl | Ph | 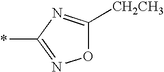 | 388.8 |

### Example 16

### 5-Bromo-2-(4-methyl-1H-imidazol-2-yl)-3-(pyrrolidin-1-ylsulfonyl)-1H-indole

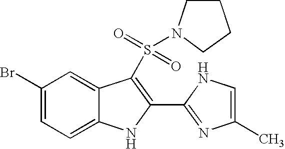

### Step 1: Ethyl 5-bromo-3-(pyrrolidin-1-ylsulfonyl)-1H-indole-2-carboxylate

Pyrrolidine (1820 uL, 21.0 mmol) was added to a solution of ethyl 5-bromo-3-(chlorosulfonyl)-1-(phenylsulfonyl)-1H-indole-2-carboxylate (3.57 g, 7.0 mmol) and pyridine (1400 μL, 14 mmol) in DCM (50 mL) at 0° C. with stirring. The resultant mixture solution was stirred from 0° C. to room temperature for 16 hours. After this time, the solution was diluted with DCM (50 mL) and washed with 1N HCl (3×50 mL), brine (50 mL), dried over Na₂SO₄, filtered, and concentrated. The concentrated residue was purified by LCMS to give the desired product ethyl 5-bromo-3-(pyrrolidin-1-ylsulfonyl)-1H-indole-2-carboxylate as a slightly yellow solid. Analytical LCMS: single peak (214 nm), 3.273 min, ES MS (M+1)=401.

### Step 2: [5-Bromo-3-(pyrrolidin-1-ylsulfonyl)-1H-indol-2-yl]methanol

LAH (1M in THF, 6.0 mL, 8.0 mmol) was added to a solution of ethyl 5-bromo-3-(pyrrolidin-1-ylsulfonyl)-1H-indole-2-carboxylate (1.61 g, 4.0 mmol) in THF (8 mL) at 0° C. with stirring. The resulting solution was stirred for 20 min at 0° C. and then added to cold 1N HCL (40 mL) dropwise to quench the reaction and excess LAH. The resultant mixture was extracted with DCM (4×80 mL). The combined DCM extracts were washed with brine (80 mL), dried over Na₂SO₄, filtered and concentrated to afford the desired product as a white solid. Analytical LCMS: single peak (214 nm), 2.861 min, ES MS (M+1)=359.

### Step 3: 5-Bromo-3-(pyrrolidin-1-ylsulfonyl)-1H-indole-2-carbaldehyde

A mixture of [5-bromo-3-(pyrrolidin-1-ylsulfonyl)-1H-indol-2-yl]methanol (1.11 g, 3.1 mmol) and MnO₂ (1.0 g, excess) in DCM (60 mL) was stirred for 5 hours at room temperature. After this time, LCMS indicated that the reaction was completion. The reaction mixture was filtered through a celite pad and washed with DCM (3×40 mL). The collected DCM solution was concentrated down to give the desired product 5-bromo-3-(pyrrolidin-1-ylsulfonyl)-1H-indole-2-carbaldehyde as slightly yellow solid Analytical LCMS: single peak (214 nm), 3.174 min, ES MS (M+1)=357.

### Step 4: 5-Bromo-2-(4-methyl-1H-imidazol-2-yl)-3-(pyrrolidin-1-ylsulfonyl)-1H-indole

A mixture of 5-bromo-3-(pyrrolidin-1-ylsulfonyl)-1H-indole-2-carbaldehyde (107 mg, 0.3 mmol) pyrualdehyde (30% water solution, 280 μL excess), concentrated NH₄OH (400 uL, excess), and EtOH (1.6 mL) was heated in a microwave at 100° C. for 1 hour. After this time, the reaction mixture solution was concentrated and the residue was purified by LCMS to give the desired product 5-bromo-2-(4-methyl-1H-imidazol-2-yl)-3-(pyrrolidin-1-ylsulfonyl)-1H-indole as a TFA salt. Analytic single peak (214 nm), 2.494 min, ES MS (M+1)=409; ¹H NMR (500 MHz, d₆-DMSO) δ 13.10 (br, 1H), 8.07 (d, J=1.8 Hz, 1H), 7.56 (d, J=8.5 Hz, 1H), 7.48 (dd, J=8.5, 1.8 Hz, 1H), 7.37-7.31 (br, 1H), 3.11-3.16 (m, 4H), 2.32 (s, 3H), 1.65-1.60 (m, 4H); HRMS calc'd for C₁₆H₁₈BrN₄O₂S (M+H). 409.0328; found 409.03167.

### Examples 17-33

The compounds in the following table were prepared in accordance with the procedure set forth in Example 16. All of the compounds in the table were prepared as TFA salts. The compound name shown in the table is the name of the free base.

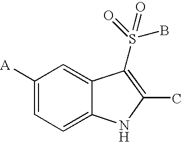

| | | | | | ES MS |
|---|---|---|---|---|---|
| Example | Name | A | B | C | (M + 1) |
| 17 | 2-[5-bromo-3-(pyrrolidin-1- ylsulfonyl)-1H-indol-2-yl]-1H- benzimidazole | Br | 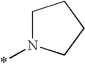 | 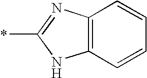 | 446.3 |
| 18 | (1S,2R,3S)-1-{2-[5-bromo-3- (pyrrolidin-1-ylsulfonyl)-1H- indol-2-yl}-1H-imidazol-4- yl}butane-1,2,3,4-tetrol | Br | 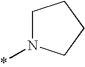 | 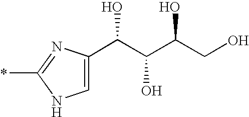 | 516.4 |
| 19 | 5-bromo-2-[4-(4-morpholin-4- ylphenyl)-1H-imidazol-2-yl]-3- (pyrrolidin-1-ylsulfonyl)-1H- indole | Br | 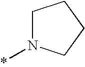 | 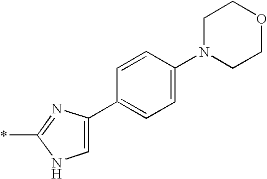 | 557.5 |
| 20 | 1-{2-[5-bromo-3-pyrrolidin-1- ylsulfonyl)-1H-indol-2-yl]-1H- imidazol-4-yl}propan-1-ol | Br | 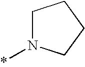 | 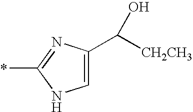 | 454.4 |
| 21 | 5-bromo-2-[4-(1- methoxypropyl)-1H-imidazol-2- yl]-3-(pyrrolidin-1-ylsulfonyl)- 1H-indole | Br | 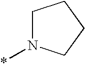 | 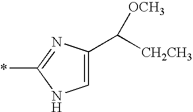 | 468.4 |
| 22 | 5-bromo-2-[4-(2,4- difluorophenyl)-1H-imidazol-2- yl]-3-(pyrrolidin-1-ylsulfonyl)- 1H-indole | Br | 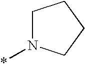 | 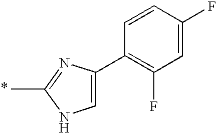 | 508.4 |
| 23 | 5-bromo-2-(4-phenyl-1H- imidazol-2-yl)-3-(pyrrolidin-1- ylsulfonyl)-1H-indole | Br | 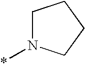 | 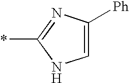 | 472.4 |
| 24 | 5-bromo-2-[4-(4-chlorophenyl)- 1H-imidazol-2-yl]-3-(pyrrolidin- 1-ylsulfonyl)-1H-indole | Br | 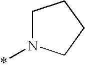 | 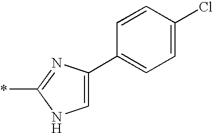 | 506.8 |
| 25 | 5-bromo-2-[4-(4-fluorophenyl)- 1H-imidazol-2-yl]-3-(pyrrolidin- 1-ylsulfonyl)-1H-indole | Br | 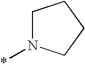 | 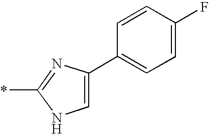 | 490.4 |
| 26 | 5-bromo-2-[4-(3,4- difluorophenyl)-1H-imidazol-2- yl]-3-(pyrrolidin-1-ylsulfonyl)- 1H-indole | Br | 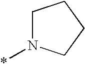 | 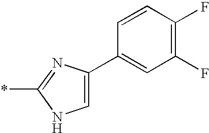 | 508.4 |
| 27 | 4-{2-[5-bromo-3-(pyrrolidin-1- ylsulfonyl)-1H-indol-2-yl]-1H- imidazol-4-yl}phenol | Br | 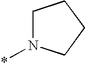 | 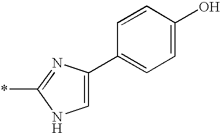 | 488.4 |
| 28 | 5-bromo-2-[4-(4- methoxyphenyl)-1H-imidazol-2- yl]-3-(pyrrolidin-1-ylsulfonyl)- 1H-indole | Br | 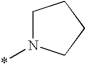 | 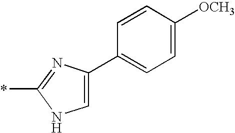 | 502.4 |
| 29 | 5-bromo-3-(pyrrolidin-1- ylsulfonyl)-2-[4-(2-thienyl)-1H- imidazol-2-yl]-1H-indole | Br | 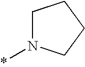 | 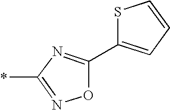 | 478.4 |
| 30 | 2-[4-(1,3-benzodioxol-5-yl)-1H- imidazol-2-yl]-5-bromo-3- (pyrrolidin-1-ylsulfonyl)-1H- indole | Br | 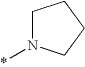 | 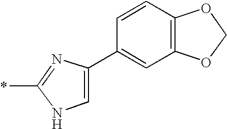 | 516.4 |
| 31 | methyl 5-{2-[5-bromo-3- (pyrrolidin-1-ylsulfonyl)-1H- indol-2-yl]-1H-imidazol-4-yl}-2- hydroxybenzoate | Br | 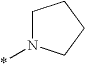 | 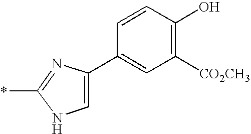 | 546.4 |
| 32 | 5-bromo-2-[4-(4-nitrophenyl)- 1H-imidazol-2-yl]-3-(pyrrolidin- 1-ylsulfonyl)-1H-indole | Br | 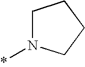 | 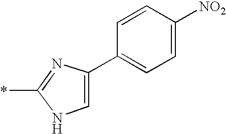 | 517.4 |
| 33 | 4-{2-[5-bromo-3-(pyrrolidin-1- ylsulfonyl)-1H-indol-2-yl]-1H- imidazol-4-yl}benzonitrile | Br | 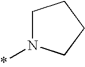 | 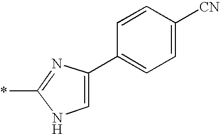 | 497.4 |

### Example 34

### 5-Chloro-3-[(cyclobutylmethyl)sulfonyl]-2-(5-methyl-1H-imidazol-2-yl)-1H-indole

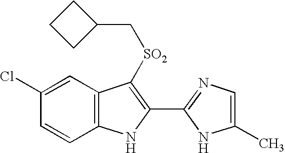

### Step 1: 5-Chloro-3-[(cyclobutylmethyl)sulfonyl]-2-hydroxymethyl-1H-indole

Ethyl 5-chloro-3-(cyclobutylmethylsulfonyl)-1-H-indole-2-carboxylate (134 mg, 0.377 mmol) was dissolved in tetrahydrofuran (1 mL) and added to a solution of lithium aluminum hydride in tetrahydrofuran (1.13 mL, 1 M) at 0° C. After stirring 30 min, the reaction was sequentially quenched with water (0.1 mL), 10% sodium hydroxide (0.17 mL) and water (0.30 mL). After stirring 30 minutes, the reaction product was filtered through celite, and the celite further washed with 10% methanol in methylene chloride. The filtrate was washed with saturated sodium chloride solution, and dried over sodium sulfate. Filtration and concentration gave the title compound.

### Step 2: 5-Chloro-3-[(cyclobutylmethyl)sulfonyl]-2-formyl-1H-indole

5-Chloro-3-[(cyclobutylmethyl)sulfonyl]-2-hydroxymethyl-1H-indole (111 mg, 0.354 mmol) was dissolved in methylene chloride (2 mL). Manganese (IV) oxide (200 mg, 2.25 mmol) was added and the reaction mixture stirred at 20° C. for 2 hours. The reaction was filtered through celite and concentrated to give the title compound.

### Step 3: 5-Chloro-3-[(cyclobutylmethyl)sulfonyl]-2-(5-methyl-1H-imidazol-2-yl)-1H-indole

5-Chloro-3-[(cyclobutylmethyl)sulfonyl]-2-formyl-1H-indole (110 mg, 0.353 mmol) was suspended in ethanol (1.6 mL) and 2-oxopropanal (0.28 mL, 40% solution in water) and concentrated ammonium hydroxide (0.40 mL) were added. The reaction was heated in a microwave for 1 hour at 100° C. The solvent was evaporated and the crude product purified by reverse phase HPLC (C18 150×21 mm column, gradient elution with 0.1% trifluoroacetic acid/water and 0.1% trifluoroacetic acid/acetonitrile). Pure fractions were combined and the solvent evaporated to give the title compound as the trifluoroacetate salt. MS (M+1)=364.08

### Examples 35-37

The compounds in the following table were prepared in accordance with the procedure set forth in Example 34. All of the compounds in the table were prepared as TFA salts. The compound name shown in the table is the name of the free base.

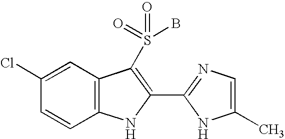

| | | | MS |
|---|---|---|---|
| Example | Name | B | (M + 1) |
| 35 | 5-chloro-3- [(cyclopentyl)sulfonyl]-2-(5- methyl-1H-imidazol-2-yl)- 1H-indole | 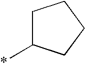 | 364.1 |
| 36 | 5-chloro-3-[(2- | CH₂CH₂CH(CH₃)₂ | 366.1 |
| | methylbutyl)sulfonyl]-2-(5- | | |
| | methyl-1H-imidazol-2-yl)- | | |
| | 1H-indole | | |
| 37 | 5-chloro-3-[(pent-3- | CH(CH₂CH₃)₂ | 366.1 |
| | yl)sulfonyl]-2-(5-methyl-1H- | | |
| | imidazol-2-yl)-1H-indole | | |

### Example 38

### Encapsulated Oral Compositions

A capsule formulation suitable for use in the present invention can be prepared by filling standard two-piece gelatin capsules each with 100 mg of the compound of Example 1, 150 mg of lactose, 50 mg of cellulose, and 3 mg of stearic acid. Encapsulated oral compositions containing any one of the compounds of Examples 2 to 37 can be similarly prepared.

### Example 39

### Assay for Inhibition of HIV Reverse Transcriptase

An assay to determine the in vivo inhibition of HIV reverse transcriptase by compounds of the present invention was conducted as follows: HIV-1 RT enzyme (1 nM) was combined with inhibitor or DMSO (10%) in assay buffer (50 mM Tris-HCl, pH 7.8, 1 mM dithiothreitol, 6 mM MgCl₂, 80 mM KCl, 0.025% CHAPS, 0.1 mM EGTA), and the mixture preincubated for 30 minutes at room temperature in microliter Optiplates (Packard). 100 μL reaction mixtures were initiated with a combination of primer-template substrate (10 nM final concentration) and dNTPs (0.6 μM dNTPs, 0.75 μM [³H]-dGTP). The heterodimeric nucleic acid substrate was generated by annealing the DNA primer pD500 (described in Shaw-Reid et al., J. Biol. Chem., 278: 2777-2780; obtained from Integrated DNA Technologies) to t500, a 500 nucleotide RNA template created by in vitro transcription (see Shaw-Reid et al., J. Biol. Chem., 278: 2777-2780). After 1 hour incubation at 37° C., reactions were quenched by 10 μL streptavidin scintillation proximity assay beads (10 mg/mL, from Amersham Biosciences) in 0.5 M EDTA, pH 8. Microtiter plates were incubated an additional 10 minutes at 37° C. prior to quantification via Topcount (Packard). Representative compounds of the present invention exhibit inhibition of the reverse transcriptase enzyme in this assay. For example, the compounds set forth above in Examples 1 to 37 were tested in the assay and all were found to have IC₅₀ values of less than 1 micromolar, except for the compound of Example 2 which had an IC₅₀ value of 4 micromolar.

Analogous assays were conducted substituting mutant HIV strains to determine the in vivo inhibition of compounds of the present invention against mutant HIV reverse transcriptase. In one strain the reverse transcriptase has the Y181C mutation and in the other strain the reverse transcriptase has the K103N mutation. The mutations were generated with the QUIKCHANGE site-directed mutagenesis kit (Stratagene). Certain compounds of the present invention exhibit inhibition of the reverse transcriptase enzyme in these assays. For example, in the Y181C mutant assay the compounds set forth above in Examples 16, 17 and 34-37 were found to have IC₅₀ values of less than 1 micromolar, and the compounds of Examples 10, 18, 20, 21, 27-30 and 32 were found to have IC₅₀ values of greater than 1 micromolar and less than 20 micromolar. The compounds of Examples 1, 3-9, 11-15, 19, 22-26, 31 and 33 were tested in the Y181C assay up to 20 micromolar, but specific IC₅₀ values were not obtained; i.e., the IC₅₀ values were greater than 20 micromolar. The compound of Example 2 was not tested in the Y181C assay. In the K103N mutant assay, the compounds of Examples 16-33 and 35 were found to have IC₅₀ values of less than 1 micromolar. The compounds of Examples 1 and 3-15 were tested in the K103N assay up to 20 micromolar, but specific IC₅₀ values were not obtained; i.e., the IC₅₀ values were greater than 20 micromolar. Specific IC₅₀ values were not obtained for the compounds of Examples 34, 36 and 37 either, but it was determined that the IC₅₀ values of these compounds were greater than 3, 1 and 10 micromolar respectively. The compound of Example 2 was not tested in the K103N assay.

### Example 40

### Assay for Inhibition of HIV Replication

An assay for the inhibition of acute HIV infection of T-lymphoid cells (alternatively referred to herein as the “spread assay”) was conducted in accordance with Vacca, J. P. et al., Proc. Natl. Acad. Sci. USA 1994, 91: 4096. Representative compounds of the present invention exhibit inhibition of HIV replication in this assay. For example, the compounds set forth in Examples 1, 5-7, 9-21 and 23-37 were found to have IC₉₅ values of less than 1 micromolar. The compound of Example 8 was found to have an IC₉₅ value of 2.5 micromolar in the spread assay. The compounds of Examples 2-4 and 22 were not tested.

### Example 41

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention that were tested in the spread assay (see Example 40) were examined for cytotoxicity. For those compounds for which an IC₉₅ value was determined in the spread assay, no cytotoxicity was exhibited at the IC₉₅ concentration; i.e., their toxicity value is greater than their IC₉₅ value. In particular, the compounds set forth in Examples 1, 5-21 and 23-37 exhibited no cytotoxicity at their IC₉₅ concentrations.

While the foregoing specification teaches the principles of the present invention, with examples provided for the purpose of illustration, the practice of the invention encompasses all of the usual variations, adaptations and/or modifications that come within the scope of the following claims.

1. A compound of Formula I, or a pharmaceutically acceptable salt thereof: 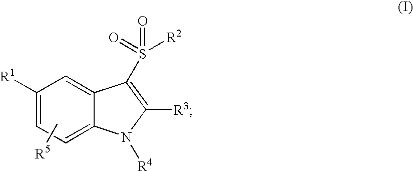
wherein:
R¹ is:
(1) halogen,
(2) CN,
(3) NO₂,
(4) C(O)R^{A},
(5) C(O)OR^{A},
(6) C(O)N(R^{A})R^{B},
(7) SR^{A},
(8) S(O)R^{A},
(9) S(O)₂R^{A},
(10) S(O)₂N(R^{A})R^{B},
(11) N(R^{A})R^{B},
(12) N(R^{A})S(O)₂R^{B},
(13) N(R^{A})C(O)R^{B},
(14) N(R^{A})C(O)OR^{B},
(15) N(R^{A})S(O)₂N(R^{A})R^{B},
(16) OC(O)N(R^{A})R^{B},
(17) N(R^{A})C(O)N(R^{A})R^{B},
(18) C₁₋₆ alkyl,
(19) C₁₋₆ haloalkyl,
(20) C₂₋₆ alkenyl,
(21) C₂₋₆ alkynyl,
(22) OH,
(23) O—C₁₋₆ alkyl,
(24) O—C₁₋₆ haloalkyl,
(25) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(26) CycA,
(27) AryA,
(28) HetA,
(29) HetR,
(30) C₁₋₆ alkyl substituted with CycA, AryA, HetA, or HetR,
(31) J-CycA,
(32) J-AryA,
(33) J-HetA, or
(34) J-HetR;
J is:
(1) O,
(2) S,
(3) S(O),
(4) S(O)₂,
(5) O—C₁₋₆ alkylene,
(6) S—C₁₋₆ alkylene,
(7) S(O)—C₁₋₆ alkylene,
(8) S(O)₂—C₁₋₆ alkylene,
(9) N(R^{A}),
(10) N(R^{A})—C₁₋₆ alkylene,
(11) C(O),
(12) C(O)—C₁₋₆ alkylene,
(13) C(O)—C₁₋₆ alkylene-O,
(14) C(O)N(R^{A}),
(15) C(O)N(R^{A})—C₁₋₆ alkylene,
(16) C(O)N(R^{A})—C₁₋₆ alkylene-C(O)O, or
(17) C(O)N(R^{A})S(O)₂;
CycA is C₃₋₈ cycloalkyl which is optionally substituted with a total of from 1 to 6 substituents, wherein:
(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl, or
(7) O—C₁₋₆ haloalkyl, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, O-HetE, or HetF;
AryA is aryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) S(O)₂R^{A},
(19) S(O)₂N(R^{A})R^{B},
(20) N(R^{A})S(O)₂R^{B},
(21) N(R^{A})S(O)₂N(R^{A})R^{B},
(22) N(R^{A})C(O)R^{B},
(23) N(R^{A})C(O)N(R^{A})R^{B},
(24) N(R^{A})C(O)—C(O)N(R^{A})R^{B}, or
(25) N(R^{A})CO₂R^{B}, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, O-HetE, or HetF;
HetA is heteroaryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) C₁₋₆ alkyl substituted with from 2 to 4 OH,
(4) O—C₁₋₆ alkyl,
(5) C₁₋₆ haloalkyl,
(6) O—C₁₋₆ haloalkyl,
(7) OH,
(8) oxo,
(9) halogen,
(10) CN,
(11) NO₂,
(12) N(R^{A})R^{B},
(13) C(O)N(R^{A})R^{B},
(14) C(O)R^{A},
(15) C(O)—C₁₋₆ haloalkyl,
(16) C(O)OR^{A},
(17) OC(O)N(R^{A})R^{B},
(18) SR^{A},
(19) S(O)R^{A},
(20) S(O)₂R^{A},
(21) S(O)₂N(R^{A})R^{B},
(22) N(R^{A})S(O)₂R^{B},
(23) N(R^{A})S(O)₂N(R^{A})R^{B},
(24) N(R^{A})C(O)R^{B},
(25) N(R^{A})C(O)N(R^{A})R^{B},
(26) N(R^{A})C(O)—C(O)N(R^{A})R^{B}, or
(27) N(R^{A})CO₂R^{B}, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, O-HetE, or HetF;
HetR is (i) a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂ or (ii) a 6- to 10-membered saturated or mono-unsaturated, bridged or fused heterobicyclic ring containing from 1 to 4 heteroatoms independently selected from N, O and S, where each S is optionally oxidized to S(O) or S(O)₂; and wherein the saturated or mono-unsaturated heterocyclic or heterobicyclic ring is optionally substituted with a total of from 1 to 4 substituents, wherein:
(i) from zero to 4 substituents are each independently halogen, CN, C₁₋₆ alkyl, OH, oxo, C(O)R^{A}, CO₂R^{A}, S(O)R^{A}, SR^{A}, S(O)₂R^{A}, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, AryE, HetE, HetF, or C₁₋₆ alkyl substituted with CycE, AryE, HetE, or HetF;
R² is:
(1) C₁₋₆ alkyl,
(2) C₁₋₆ haloalkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B},
(3) CycB,
(4) AryB,
(5) HetB,
(6) HetS,
(7) C₁₋₆ alkyl substituted with CycB, AryB, HetB, or HetS,
(8) N(R^{A})—C₁₋₆ alkyl,
(9) N(R^{A})—C₁₋₆ alkyl, wherein the alkyl is substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(10) N(R^{A})-CycB,
(11) N(R^{A})-AryB,
(12) N(R^{A})-HetB, or
(13) N(R^{A})—C₁₋₆ alkyl, wherein the alkyl is substituted with CycB, AryB, HetB, or HetS;
CycB independently has the same definition as CycA;
AryB independently has the same definition as AryA;
HetB independently has the same definition as HetA;
HetS independently has the same definition as HetR;
R³ is HetC, wherein HetC independently has the same definition as HetA;
R⁴ is H, C₁₋₆ alkyl, C(O)C₁₋₆ alkyl, C(O)-CycD, C(O)-AryD, C(O)-HetD, or C(O)HetU;
CycD independently has the same definition as CycA;
AryD independently has the same definition as AryA;
HetD independently has the same definition as HetA;
HetU independently has the same definition as HetR;
R⁵ is H or independently has the same definition as R¹;
each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
each CycE is independently C₃₋₈ cycloalkyl which is optionally substituted with a total of from 1 to 4 substituents, wherein:
(i) from zero to 4 substituents are each independently halogen, C₁₋₆ alkyl, OH, O—C₁₋₆ alkyl, C₁₋₆ haloalkyl, or O—C₁₋₆ haloalkyl, and
(ii) from zero to 2 substituents are each independently CycG, AryG, HetG, HetH, or C₁₋₆ alkyl substituted with CycG, AryG, O-AryG, HetG, or HetH;
each AryE is independently phenyl or naphthyl, wherein the phenyl or naphthyl is optionally substituted with a total of from 1 to 5 substituents, wherein:
(i) from zero to 5 substituents are each independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B}, and
(ii) from zero to 2 substituents are each independently CycG, AryG, HetG, HetH, or C₁₋₆ alkyl substituted with CycG, AryG, O-AryG, HetG, or HetH;
each HetE is independently (i) a 5- or 6-membered heteroaromatic ring containing from 1 to 4 heteroatoms independently selected from N, O and S, wherein each N is optionally in the form of an oxide, or (ii) a 9- or 10-membered fused heterobicyclic ring selected from 2,3-dihydrobenzo-1,4-dioxinyl and benzo-1,3-dioxolyl; and wherein the heteroaromatic ring or the heterobicyclic ring is optionally substituted with a total of from 1 to 4 substituents wherein:
(i) from zero to 4 substituents are each independently halogen, C₁₋₆ alkyl, C₁₋₆ haloalkyl, O—C₁₋₆ alkyl, O—C₁₋₆ haloalkyl, OH, C(O)R^{A}, CO₂R^{A}, SO₂R^{A}, N(R^{A})R^{B}, N(R^{A})C(O)N(R^{A})R^{B}, or N(R^{A})CO₂R^{B}, and
(ii) from zero to 2 substituents are each independently CycG, AryG, HetG, HetH, or C₁₋₆ alkyl substituted with CycG, AryG, O-AryG, HetG, or HetH;
each HetF is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, and wherein the saturated or mono-unsaturated heterocyclic ring is optionally substituted with a total of from 1 to 4 substituents, wherein:
(i) from zero to 4 substituents are each independently halogen, CN, C₁₋₆ alkyl, OH, oxo, O—C₁₋₆ alkyl, C₁₋₆ haloalkyl, O—C₁₋₆ haloalkyl, C(O)R^{A}, CO₂R^{A}, or SO₂R^{A}, and
(ii) from zero to 2 substituents are each independently CycG, AryG, HetG, HetH, or C₁₋₆ alkyl substituted with CycG, AryG, O-AryG, HetG, or HetH;
each CycG is independently C₃₋₈ cycloalkyl which is optionally substituted with from 1 to 4 substituents, each of which is independently halogen, C₁₋₆ alkyl, OH, O—C₁₋₆ alkyl, C₁₋₆ haloalkyl, or O—C₁₋₆ haloalkyl;
each AryG is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
each HetG is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, OH, C(O)R^{A}, CO₂R^{A}, SO₂R^{A}, N(R^{A})R^{B}, N(R^{A})C(O)N(R^{A})R^{B}, or N(R^{A})CO₂R^{B};
each HetH is independently a 4- to 7-membered, saturated or mono-unsaturated heterocyclic ring containing at least one carbon atom and from 1 to 4 heteroatoms independently selected from N, O and S, where each S is optionally oxidized to S(O) or S(O)₂, and wherein the saturated or mono-unsaturated heterocyclic ring is optionally substituted with from 1 to 4 substituents, each of which is independently halogen, CN, C₁₋₆ alkyl, OH, oxo, O—C₁₋₆ alkyl, C₁₋₆ haloalkyl, O—C₁₋₆ haloalkyl, C(O)R^{A}, CO₂R^{A}, or SO₂R^{A};
each R^{A} is independently H or C₁₋₆ alkyl; and
each R^{B} is independently H or C₁₋₆ alkyl;
and with the proviso that:
(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

2. The compound according to claim 1, or a pharmaceutically acceptable salt thereof, wherein R¹ is:
(1) halogen,
(2) CN,
(3) NO₂,
(4) N(R^{A})R^{B},
(5) N(R^{A})S(O)₂R^{B},
(6) N(R^{A})C(O)R^{B},
(7) C₁₋₆ alkyl,
(8) C₁₋₆ haloalkyl,
(9) C₂₋₆ alkenyl,
(10) OH,
(11) O—C₁₋₆ alkyl,
(12) O—C₁₋₆ haloalkyl,
(13) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(14) CycA,
(15) AryA,
(16) HetA, or
(17) C₁₋₆ alkyl substituted with CycA, AryA, or HetA; and
R⁵ is H;
and with the proviso that:
(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

3. The compound according to claim 1, or a pharmaceutically acceptable salt thereof, wherein R² is AryB or HetS; and with the proviso that:
(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

4. The compound according to claim 3, or a pharmaceutically acceptable salt thereof, wherein:
AryB is phenyl, wherein the phenyl is optionally substituted with a total of from 1 to 5 substituents, each of which is independently:
(1) C₁₋₄ alkyl,
(2) O—C₁₋₄ alkyl,
(3) C₁₋₄ haloalkyl,
(4) O—C₁₋₄ haloalkyl,
(5) OH,
(6) halogen,
(7) CN,
(8) NO₂,
(9) NH₂,
(10) N(H)—C₁₋₄ alkyl,
(11) N(C₁₋₄ alkyl)₂,
(12) C(O)NH₂,
(13) C(O)N(H)—C₁₋₄ alkyl,
(14) C(O)N(C₁₋₄ alkyl)₂,
(15) C(O)—C₁₋₄ alkyl,
(16) CO₂—C₁₋₄ alkyl,
(17) S—C₁₋₄ alkyl,
(18) S(O)—C₁₋₄ alkyl,
(19) SO₂—C₁₋₄ alkyl,
(20) SO₂NH₂,
(21) SO₂N(H)—C₁₋₄ alkyl,
(22) SO₂N(C₁₋₄ alkyl)₂,
(23) SO₂N(H)C(O)—C₁₋₄ alkyl,
(24) SO₂N(C₁₋₄ alkyl)C(O)—C₁₋₄ alkyl,
(25) N(H)C(O)—C₁₋₄ alkyl, or
(26) N(C₁₋₄ alkyl)C(O)—C₁₋₄ alkyl; and
HetS is a 4- to 7-membered, saturated or mono-unsaturated heterocyclic ring or a 6- to 10-membered saturated or mono-unsaturated, bridged or fused heterobicyclic ring, wherein the heterocyclic or heterobicyclic ring contains a nitrogen atom which is directly attached to the rest of the molecule and optionally contains an additional heteroatom selected from N, O, and S, where the S is optionally oxidized to S(O) or S(O)₂; and wherein the heterocyclic or heterobicyclic ring is optionally substituted with a total of from 1 to 4 substituents, wherein:
(i) from zero to 4 substituents are each independently Cl, Br, F, C₁₋₄ alkyl, OH, oxo, S(O)₂—C₁₋₄ alkyl, O—C₁₋₄ alkyl, O—C₁₋₄ haloalkyl, or C₁₋₄ haloalkyl; and
(ii) from zero to 1 substituent is AryE, HetE, CH₂-AryE, or CH₂-HetE;
and with the proviso that:
(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, C₁₋₄ alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

5. The compound according to claim 1, or a pharmaceutically acceptable salt thereof, wherein R³ is HetC; and HetC is:
a 5-membered heteroaromatic ring containing from 1 to 3 heteroatoms independently selected from 1 to 3N atoms, from zero to 1 O atom, and from zero to 1 S atom, wherein the heteroaromatic ring is connected to the rest of the molecule via a ring carbon, and the heteroaromatic ring is optionally substituted with from 1 to 2 substituents each of which is independently
(1) C₁₋₄ alkyl,
(2) C₁₋₄ alkyl substituted with OH or O—C₁₋₄ alkyl,
(3) C₁₋₄ alkyl substituted with from 2 to 4 OH,
(4) O—C₁₋₄ alkyl,
(5) C₁₋₄ haloalkyl,
(6) O—C₁₋₄ haloalkyl,
(7) OH,
(8) Cl, Br, or F,
(9) CN,
(10) C(O)N(H)—C₁₋₄ alkyl,
(11) C(O)N(C₁₋₄ alkyl)₂,
(12) S(O)₂—C₁₋₄ alkyl,
(13) S(O)₂NH₂,
(14) S(O)₂N(H)—C₁₋₄ alkyl,
(15) S(O)₂N(C₁₋₄ alkyl)₂,
(16) CycE, AryE, or HetE, or
(17) CH₂-CycE, CH₂-AryE, CH₂—O-AryE, or CH₂-HetE, or
(ii) 5-membered heteroaromatic ring containing from 1 to 2 heteroatoms independently selected from 1 to 2 N atoms, from zero to 1 O atom, and from zero to 1 S atom, wherein the heteroaromatic ring is connected to the rest of the molecule via a ring carbon and has fused thereto a benzene ring wherein the benzene ring is optionally substituted with from 1 to 3 substituents each of which is independently
(1) C₁₋₄ alkyl,
(2) O—C₁₋₄ alkyl,
(3) C₁₋₄ haloalkyl,
(4) O—C₁₋₄ haloalkyl,
(5) OH,
(6) Cl, Br, or F,
(7) CN,
(8) C(O)N(H)—C₁₋₄ alkyl,
(9) C(O)N(C₁-4 alkyl)₂,
(10) S(O)₂—C₁₋₄ alkyl,
(11) S(O)₂NH₂,
(12) S(O)₂N(H)—C₁₋₄ alkyl, or
(13) S(O)₂N(C₁₋₄ alkyl)₂;
and with the proviso that:
(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then R³ is not a 5-membered heteroaromatic ring containing from 1 to 3 heteroatoms independently selected from 1 to 3N atoms, from zero to 1 O atom, and from zero to 1 S atom, wherein the heteroaromatic ring is connected to the rest of the molecule via a ring carbon and wherein the heteroaromatic ring is unsubstituted or substituted with one or more substituents each of which is independently C₁₋₄ alkyl, Cl, Br, F, S(O)₂NH₂, CN, C₃₋₅ cycloalkyl, or C₁₋₄ haloalkyl having from 1 to 3 halogen substituents.

6. The compound according to claim 5, or a pharmaceutically acceptable salt thereof, wherein:
R³ is 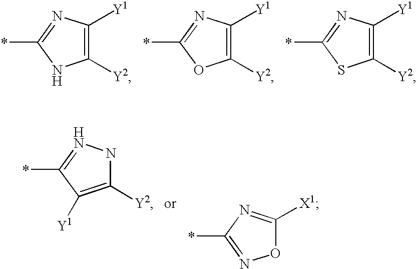
X¹ is:
(1) H,
(2) C₁₋₄ alkyl,
(3) C₁₋₄ alkyl substituted with OH or O—C₁₋₄ alkyl,
(4) C₁₋₄ alkyl substituted with from 2 to 4 OH,
(5) C₃₋₆ cycloalkyl which is optionally substituted with C₁₋₄ alkyl or phenyl,
(6) phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂,
(7) phenyl substituted with a heterocyclic ring selected from the groun consisting of: 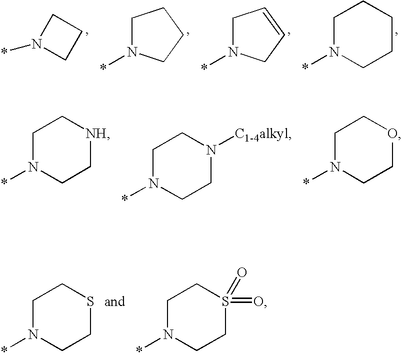
wherein the asterisk denotes the point of attachment to the rest of the molecule,
(8) CH₂-phenyl,
(9) CH₂—O-phenyl,
(10) heteroaryl selected from the group consisting of pyrrolyl, imidazolyl, furanyl, thienyl, oxazolyl, thiazolyl, pyridinyl, pyrimidinyl, and pyrazinyl, wherein the heteroaryl is optionally substituted with from 1 to 3 substituents each of which is independently Cl, Br, F, C₁₋₄ alkyl, CF₃, OH, O—C₁₋₄ alkyl, or OCF₃, or
(11) heteroaryl selected from the group consisting of 2,3-dihydrobenzo-1,4-dioxinyl and benzo-1,3-dioxolyl;
Y¹ independently has the same definition as X¹; and
Y² independently has the same definition as X¹;
or alternatively, Y¹ and Y² together with the carbon atoms to which each is attached form a benzo ring;
and with the proviso that:
(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, R⁴ is H, and R⁵ is H, then (i) Xl in the definition of R³ is not H, C₁₋₄ alkyl, or C₃₋₅ cycloalkyl and (ii) one of Y¹ and Y² in the definition of R³ is not H, C₁₋₄ alkyl, or C₃₋₅ cycloalkyl when the other of Y¹ and Y² is H, C₁₋₄ alkyl, or C₃₋₅ cycloalkyl.

7. The compound according to claim 1, or a pharmaceutically acceptable salt thereof, wherein R⁴ is H;
and with the proviso that:
(A) when R¹ is halogen, R² is AryB and AryB is unsubstituted phenyl or phenyl substituted with from 1 to 5 substituents each of which is independently halogen, NO₂, CN, C₁₋₄ alkyl, O—C₁₋₄ alkyl, C₁₋₄ alkylamino, sulfonamido, or C₁₋₄ haloalkyl having from 1 to 3 halogen substituents, and R⁵ is H, then R³ is not (i) a 5- or 6-membered heteroaromatic ring containing from 1 to 4 heteroatoms independently selected from N, O and S, wherein each N is optionally in the form of an oxide, and wherein the heteroaromatic ring is unsubstituted or substituted with one or more substituents each of which is independently amino, C₁₋₄ alkyl, alkylamino, halogen, sulfonamido, CN, C₃₋₅ cycloalkyl, or C₁₋₄ haloalkyl having from 1 to 3 halogen substituents or (ii) 4,5,6,7-hexahydrobenzimidazol-2-yl.

8. The compound according to claim 1, or a pharmaceutically acceptable salt thereof, wherein:
R¹ is halogen;
R² is:
(i) phenyl, wherein the phenyl is optionally substituted with a total of from 1 to 3 substituents, each of which is independently:
(1) C₁₋₄ alkyl,
(2) O—C₁₋₄ alkyl,
(3) C₁₋₄ haloalkyl,
(4) O—C₁₋₄ haloalkyl,
(5) OH,
(6) halogen,
(7) CN,
(8) NO₂,
(9) NH₂,
(10) N(H)—C₁₋₄ alkyl,
(11) N(C₁₋₄ alkyl)₂,
(12) C(O)NH₂,
(13) C(O)N(H)—C₁₋₄ alkyl,
(14) C(O)N(C₁₋₄ alkyl)₂,
(15) C(O)—C₁₋₄ alkyl,
(16) CO₂—C₁₋₄ alkyl,
(17) S—C₁₋₄ alkyl,
(18) S(O)—C₁₋₄ alkyl,
(19) SO₂—C₁₋₄ alkyl,
(20) SO₂NH₂,
(21) SO₂N(H)—C₁₋₄ alkyl,
(22) SO₂N(C₁₋₄ alkyl)₂,
(23) SO₂N(H)C(O)—C₁₋₄ alkyl,
(24) SO₂N(C₁₋₄ alkyl)C(O)—C₁₋₄ alkyl,
(25) N(H)C(O)—C₁₋₄ alkyl, or
(26) N(C₁₋₄ alkyl)C(O)—C₁₋₄ alkyl, or
(ii) HetS, wherein HetS is a 5- or 6-membered, saturated or mono-unsaturated heterocyclic ring containing a nitrogen atom that is directly attached to the rest of the molecule and optionally containing an additional heteroatom selected from N, O, and S, where the S is optionally oxidized to S(O) or S(O)₂; and wherein the heterocyclic ring is optionally substituted with a total of from 1 to 3 substituents, each of which is independently Cl, Br, F, C₁₋₄ alkyl, OH, oxo, S(O)₂—C₁₋₄ alkyl, O—C₁₋₄ alkyl, O—C₁₋₄ haloalkyl, or C₁₋₄ haloalkyl;
R₃ is:
(i) a 5-membered heteroaromatic ring containing from 1 to 3 heteroatoms independently selected from 1 to 3 N atoms, from zero to 1 O atom, and from zero to 1 S atom, wherein the heteroaromatic ring is connected to the rest of the molecule via a ring carbon, and the heteroaromatic ring is optionally substituted with from 1 to 2 substituents each of which is independently:
(1) C₁₋₄ alkyl,
(2) C₁₋₄ alkyl substituted with OH or O—C₁₋₄ alkyl,
(3) C₁₋₄ alkyl substituted with from 2 to 4 OH,
(4) O—C₁₋₄ alkyl,
(5) C₁₋₄ haloalkyl,
(6) O—C₁₋₄ haloalkyl,
(7) OH,
(8) Cl, Br, or F,
(9) CN,
(10) C(O)N(H)—C₁₋₄ alkyl,
(11) C(O)N(C₁₋₄ alkyl)₂,
(12) S(O)₂—C₁₋₄ alkyl,
(13) S(O)₂NH₂,
(14) S(O)₂N(H)—C₁₋₄ alkyl,
(15) S(O)₂N(C₁₋₄ alkyl)₂,
(16) CycE, AryE, or HetE, or
(17) CH₂-CycE, CH₂-AryE, CH₂—O-AryE, or CH₂-HetE, or
(ii) 5-membered heteroaromatic ring containing from 1 to 2 heteroatoms independently selected from 1 to 2N atoms, from zero to 1 O atom, and from zero to 1 S atom, wherein the heteroaromatic ring is connected to the rest of the molecule via a ring carbon and has fused thereto a benzene ring wherein the benzene ring is optionally substituted with from 1 to 3 substituents each of which is independently
(1) C₁₋₄ alkyl,
(2) O—C₁₋₄ alkyl,
(3) C₁₋₄ haloalkyl,
(4) O—C₁₋₄ haloalkyl,
(5) OH,
(6) Cl, Br, or F,
(7) CN,
(8) C(O)N(H)—C₁₋₄ alkyl,
(9) C(O)N(C₁₋₄ alkyl)₂;
(10) S(O)₂—C₁₋₄ alkyl,
(11) S(O)₂NH₂;
(12) S(O)₂N(H)—C₁₋₄ alkyl, or
(13) S(O)₂N(C₁₋₄ alkyl)₂;
each CycE is independently C₃₋₆ cycloalkyl which is optionally substituted with a total of from 1 to 3 substituents, wherein:
(i) from zero to 3 substituents are each independently C₁₋₄ alkyl, OH, or O—C₁₋₄ alkyl, and
(ii) from zero to 1 substituent is phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂;
each AryE is independently phenyl, which is optionally substituted with a total of from 1 to 3 substituents, wherein:
(i) from zero to 3 substituents are each independently C₁₋₄ alkyl, O—C₁₋₄ alkyl, C₁₋₄ fluoroalkyl, O—C₁₋₄ fluoroalkyl, OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂, and
(ii) from zero to 1 substituent is a 4- to 7-membered saturated or mono-unsaturated heterocyclic ring containing from 1 to 2 heteroatoms selected from 1 to 2N atoms, zero to 1 O atom, and zero to 1 S atom, where the S is optionally oxidized to S(O) or S(O)₂, and wherein the saturated or mono-unsaturated heterocyclic ring is optionally substituted with from 1 to 3 substituents, each of which is independently C₁₋₄ alkyl, OH, oxo, O—C₁₋₄ alkyl, C(O)—C₁₋₄ alkyl, C(O)O—C₁₋₄ alkyl, or SO₂—C₁₋₄ alkyl;
each HetE is independently (i) a 5- or 6-membered heteroaromatic ring selected from the group consisting of pyrrolyl, pyrazolyl, imidazolyl, triazolyl, tetrazolyl, furanyl, oxazolyl, isoxazolyl, thienyl, thiazolyl, isothiazolyl, oxadiazolyl, pyridinyl, pyrimidinyl, and pyrazinyl or (ii) a 9- or 10-membered fused heterobicyclic ring selected from 2,3-dihydrobenzo-1,4-dioxinyl and benzo-1,3-dioxolyl; and wherein the heteroaromatic ring or the heterobicyclic ring is optionally substituted with a total of from 1 to 3 substituents each of which is independently halogen, C₁₋₄ alkyl, C₁₋₄ fluoroalkyl, O—C₁₋₄ alkyl, O—C₁₋₄ fluoroalkyl, or OH;
R⁴ is H; and
R⁵ is H;
and with the proviso that:
(A) when R² is unsubstituted phenyl or phenyl substituted with from 1 to 3 substituents each of which is independently halogen, NO₂, CN, C₁₋₄ alkyl, O—C₁₋₄ alkyl, SO₂NH₂, or C₁₋₄ haloalkyl having from 1 to 3 halogen substituents, then R₃ is not a 5-membered heteroaromatic ring containing from 1 to 3 heteroatoms selected from 1 to 3 N atoms, from zero to 1 O atom, and from zero to 1 S atom, wherein the heteroaromatic ring is connected to the rest of the molecule via a ring carbon, and the heteroaromatic ring is unsubstituted or substituted with from 1 to 2 substituents each of which is independently C₁₋₄ alkyl, Cl, Br, F, SO₂NH₂, CN, C₃₋₅ cycloalkyl, or C₁₋₄ haloalkyl having from 1 to 3 halogen substituents.

9. The compound according to claim 8, or a pharmaceutically acceptable salt thereof, wherein:
R³ is 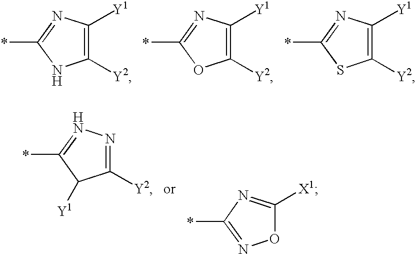
X¹ is:
(1) H,
(2) C₁₋₄ alkyl,
(3) C₁₋₄ alkyl substituted with OH or O—C₁₋₄ alkyl,
(4) C₁₋₄ alkyl substituted with from 2 to 4 OH,
(5) C₃₋₆ cycloalkyl which is optionally substituted with C₁₋₄ alkyl or phenyl,
(6) phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂,
(7) phenyl substituted with a heterocyclic ring selected from the grout) consisting of: 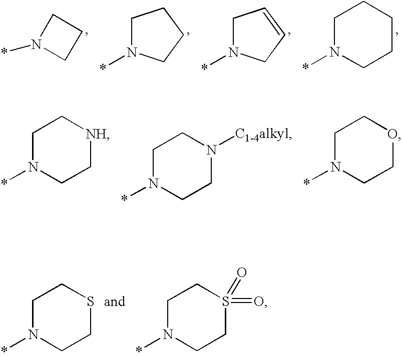
wherein the asterisk denotes the point of attachment to the rest of the molecule,
(8) CH₂-phenyl,
(9) CH₂—O-phenyl,
(10) heteroaryl selected from the group consisting of pyrrolyl, imidazolyl, furanyl, thienyl, oxazolyl, thiazolyl, pyridinyl, pyrimidinyl, and pyrazinyl, wherein the heteroaryl is optionally substituted with from 1 to 3 substituents each of which is independently Cl, Br, F, C₁₋₄ alkyl, CF₃, OH, O—C₁₋₄ alkyl, or OCF₃, or
(11) heteroaryl selected from the group consisting of 2,3-dihydrobenzo-1,4-dioxinyl and benzo-1,3-dioxolyl;
Y¹ independently has the same definition as X¹; and
Y² independently has the same definition as X¹;
or alternatively, Y¹ and Y² together with the carbon atoms to which each is attached form a benzo ring;
and with the proviso that:
(A) when R² is unsubstituted phenyl or phenyl substituted with from 1 to 3 substituents each of which is independently halogen, NO₂, CN, C₁₋₄ alkyl, O—C₁₋₄ alkyl, SO²NH², or C₁₋₄ haloalkyl having from 1 to 3 halogen substituents, then X¹ in the definition of R³ is not H, C₁₋₄ alkyl, or C₃₋₅ cycloalkyl, and one of Y¹ and Y² in the definition of R³ is not H, C₁₋₄ alkyl, or C³⁻⁵ cycloalkyl when the other of Y¹ and Y² is H, C₁₋₄ alkyl, or C₃₋₅ cycloalkyl.

10. The compound according to claim 9, or a pharmaceutically acceptable salt thereof, wherein:
R¹ is Cl or Br;
R² is:
(i) phenyl, which is optionally substituted with a total of from 1 to 3 substituents, each of which is independently CH₃, OCH₃, CF₃, OCF₃, OH, Cl, Br, F, CN, C(O)N(CH₃)₂, C(O)CH₃, CO₂CH₃, or SO₂CH₃, or
(ii) a cathrated heterocyclic ring selected from the groun consisting of: 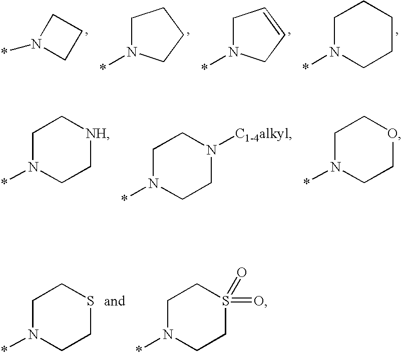
wherein the asterisk denotes the point of attachment to the rest of the molecule,
R³ is 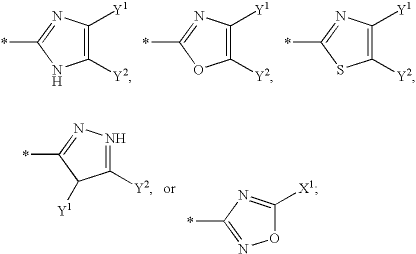
X¹ is:
(1) H,
(2) C₁₋₃ alkyl,
(3) C₁₋₃ alkyl substituted with OH or OCH₃,
(4) C₁₋₄ alkyl substituted with from 2 to 4 OH,
(5) C₃₋₆ cycloalkyl which is optionally substituted with C₁₋₄ alkyl or phenyl,
(6) phenyl which is optionally substituted with from 1 to 3 substituents each of which is independently CH₃, OCH₃, CF₃, OCF₃, OH, Cl, Br, F, CN, NO₂, C(O)N(H)CH₃, C(O)N(CH₃)₂, CO₂CH₃, or S(O)₂CH₃,
(7) phenyl substituted with a saturated heterocyclic ring selected from the group consisting of: 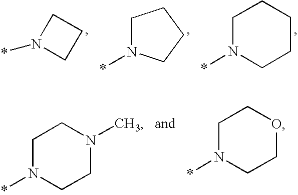
wherein the asterisk denotes the point of attachment to the rest of the molecule,
(8) CH₂-phenyl,
(9) CH₂—O-phenyl,
(10) thienyl or pyridinyl, or
(11) benzo-1,3-dioxolyl;
one of Y¹ and Y² independently has the same definition as X ¹ , and the other of Y¹ and Y² is H;
or alternatively, Y¹ and Y² together with the carbon atoms to which each is attached form a benzo ring;
and with the proviso that:
(A) when R² is unsubstituted phenyl or phenyl substituted with from 1 to 3 substituents each of which is independently CH₃, OCH₃, CF_{3,} , Cl, Br, F, or CN, then (i) X¹ in the definition of R³ is not H, C₁₋₃ alkyl, or C₃₋₅ cycloalkyl and (ii) one of Y¹ and Y² in the definition of R³ is not H, C₁₋₄ alkyl, or C₃₋₅ cycloalkyl when the other of Y¹ and Y² is H.

11. The compound according to claim 10, or a pharmaceutically acceptable salt thereof, wherein:
R² is phenyl and R³ is 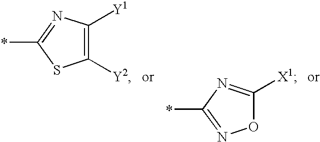
R² is 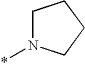
and R³ is 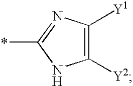
and with the proviso that:
(A) when R² is unsubstituted phenyl, then X¹ in the definition of R³ is not H, C₁₋₃ alkyl, or C₃₋₅ cycloalkyl, and one of Y¹ and Y² in the definition of R³ is (i) not H, C₁₋₃ alkyl, or C₃₋₅ cycloalkyl when the other of Y¹ and Y² is H.

12. The compound according to claim 1, or a pharmaceutically acceptable salt thereof, wherein:
R¹ is halogen;
R² is:
(1) C₁₋₆ alkyl,
(2) C₃₋₆ cycloalkyl, or
(3) C₁₋₆ alkyl substituted with C₃₋₆ cycloalkyl;
R³ is 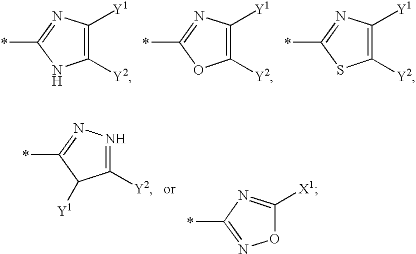
X¹ is:
(1) H,
(2) C₁₋₄ alkyl,
(3) C₁₋₄ alkyl substituted with OH or O—C₁₋₄ alkyl,
(4) C₁₋₄ alkyl substituted with from 2 to 4 OH,
(5) C₃₋₆ cycloalkyl which is optionally substituted with C₁₋₄ alkyl or phenyl,
(6) phenyl which is optionally substituted with from 1 to 3 substituents each of which is independently C₁₋₄ alkyl, O—C₁₋₄ alkyl, C₁₋₄ fluoroalkyl, O—C₁₋₄ fluoroalkyl, OH, Cl, Br, F, CN, NO₂, C(O)N(H)—C₁₋₄ alkyl, C(O)N(C₁₋₄ alkyl)₂, CO₂—C₁₋₄ alkyl, S(O)₂—C₁₋₄ alkyl, S(O)₂NH₂, S(O)₂N(H)—C₁₋₄ alkyl, or S(O)₂N(C₁₋₄ alkyl)₂,
(7) phenyl substituted with a heterocyclic ring selected from the group consisting of: 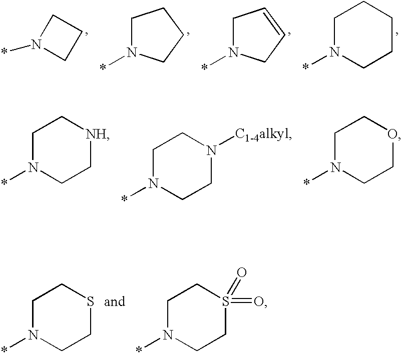
wherein the asterisk denotes the point of attachment to the rest of the molecule,
(8) CH₂-phenyl,
(9) CH₂—O-phenyl,
(10) heteroaryl selected from the group consisting of pyrrolyl, imidazolyl, furanyl, thienyl, oxazolyl, thiazolyl, pyridinyl, pyrimidinyl, and pyrazinyl, wherein the heteroaryl is optionally substituted with from 1 to 3 substituents each of which is independently Cl, Br, F, C₁₋₄ alkyl, CF₃, OH, O—C₁₋₄ alkyl, or OCF₃, or
(11) heteroaryl selected from the group consisting of 2,3-dihydrobenzo-1,4-dioxinyl and benzo-1,3-dioxolyl;
Y¹ independently has the same definition as X¹; and
Y² independently has the same definition as X¹;
or alternatively, Y¹ and Y² together with the carbon atoms to which each is attached form a benzo ring;
R⁴ is H; and
R⁵ is H.

13. The compound according to claim 12, or a pharmaceutically acceptable salt thereof, wherein:
R¹ is Cl or Br;
R² is:
(1) C₁₋₅ alkyl,
(2) C₃₋₆ cycloalkyl, or
(3) (CH₂)₁₋₂—C₃₋₆ cycloalkyl;
R³ is 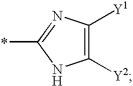
one of Y¹ and Y² is H, and the other of Y¹ and Y² is:
(1) H,
(2) C₁₋₃ alkyl,
(3) C₁₋₃ alkyl substituted with OH or OCH₃,
(4) C₁₋₄ alkyl substituted with from 2 to 4 OH,
(5) C₃₋₆ cycloalkyl which is optionally substituted with C₁₋₄ alkyl or phenyl,
(6) phenyl which is optionally substituted with from 1 to 3 substituents each of which is independently CH₃, OCH₃, CF₃, OCF₃, OH, Cl, Br, F, CN, NO₂, C(O)N(H)CH₃, C(O)N(CH₃)₂, CO₂CH_{3,} or S(O)₂CH₃,
(7) phenyl substituted with a saturated heterocyclic ring selected from the group consisting of: 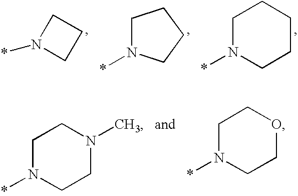
wherein the asterisk denotes the point of attachment to the rest of the molecule,
(8) CH₂-phenyl,
(9) CH₂—O-phenyl,
(10) thienyl or pyridinyl, or
(11) benzo-1,3-dioxolyl;
R⁴ is H; and
R⁵ is H.

14. A compound, or a pharmaceutically acceptable salt thereof, selected from the group consisting of:
5-chloro-3-(phenylsulfonyl)-2-(4-pyridin-2-yl-1,3-thiazol-2-yl)-1H-indole;
5-chloro-3-(phenylsulfonyl)-2-(4-pyridin-3-yl-1,3-thiazol-2-yl)-1H-indole;
5-chloro-3-(phenylsulfonyl)-2-(4-pyridin-4-yl-1,3-thiazol-2-yl)-1H-indole;
5-chloro-2-[5-(2-chlorophenyl)-1,2,4-oxadiazol-3-yl]-3-(phenylsulfonyl)-1H-indole;
5-chloro-3-(phenylsulfonyl)-2-(5-propyl-1,2,4-oxadiazol-3-yl)-1H-indole;
5-chloro-2-[5-(2-fluorophenyl)-1,2,4-oxadiazol-3-yl]-3-(phenylsulfonyl)-1H-indole;
5-chloro-2-{5-[(1R,2R)-2-phenylcyclopropyl]-1,2,4-oxadiazol-3-yl}-3-(phenylsulfonyl)-1H-indole;
5-chloro-2-[5-(phenoxymethyl)-1,2,4-oxadiazol-3-yl]-3-(phenylsulfonyl)-1H-indole;
5-chloro-3-(phenylsulfonyl)-2-(5-pyridin-4-yl-1,2,4-oxadiazol-3-yl)-1H-indole;
5-chloro-2-[5-(2,4-difluorophenyl)-1,2,4-oxadiazol-3-yl]-3-(phenylsulfonyl)-1H-indole;
5-chloro-2-(5-methyl-1,2,4-oxadiazol-3-yl)-3-(phenylsulfonyl)-1H-indole;
5-chloro-2-(5-cyclobutyl-1,2,4-oxadiazol-3-yl)-3-(phenylsulfonyl)-1H-indole;
5-chloro-2-[5-(methoxyrnethyl)-1,2,4-oxadiazol-3-yl]-3-(phenylsulfonyl)-1H-indole;
2-(5-benzyl-1,2,4-oxadiazol-3-yl)-5-chloro-3-(phenylsulfonyl)-1H-indole;
5-chloro-2-(5-ethyl-1,2,4-oxadiazol-3-yl)-3-(phenylsulfonyl)-1H-indole;
5-bromo-2-(4-methyl-1H-imidazol-2-yl)-3-(pyrrolidin-1-ylsulfonyl)-1H-indole;
2-[5-bromo-3-(pyrrolidin-1-ylsulfonyl)-1H-indol-2-yl]-1H-benzimidazole;
(1S,2R,3S)-1-{2-[5-bromo-3-(pyrrolidin-1-ylsulfonyl)-1H-indol-2-yl]-1H-imidazol-4-yl}butane-1,2,3,4-tetrol;
5-bromo-2-[4-(4-morpholin-4-ylphenyl)-1H-imidazol-2-yl]-3-(pyrrolidin-1-ylsulfonyl)-1H-indole;
1-{2-[5-bromo-3-(pyrrolidin-1-ylsulfonyl)-1H-indol-2-yl]-1H-imidazol-4-yl}propan-1-ol;
5-bromo-2-[4-(1-methoxypropyl)-1H-imidazol-2-yl]-3-(pyrrolidin-1-ylsulfonyl)-1H-indole;
5-bromo-2-[4-(2,4-difluorophenyl)-1H-imidazol-2-yl]-3-(pyrrolidin-1-ylsulfonyl)-1H-indole;
5-bromo-2-(4-phenyl-1H-imidazol-2-yl)-3-(pyrrolidin-1-ylsulfonyl)-1H-indole;
5-bromo-2-[4-(4-chlorophenyl)-1H-imidazol-2-yl]-3-(pyrrolidin-1-ylsulfonyl)-1H-indole;
5-bromo-2-[4-(4-fluorophenyl)-1H-imidazol-2-yl]-3-(pyrrolidin-1-ylsulfonyl)-1H-indole;
5-bromo-2-[4-(3,4-difluorophenyl)-1H-imidazol-2-yl]-3-(pyrrolidin-1-ylsulfonyl)-1H-indole;
4-{2-[5-bromo-3-(pyrrolidin-1-ylsulfonyl)-1H-indol-2-yl]-1H-imidazol-4-yl}phenol;
5-bromo-2-[4-(4-methoxyphenyl)-1H-imidazol-2-yl]-3-(pyrrolidin-1-ylsulfonyl)-1H-indole;
5-bromo-3-(pyrrolidin-1-ylsulfonyl)-2-[4-(2-thienyl)-1H-imidazol-2-yl]-1H-indole;
2-[4-(1,3-benzodioxol-5-yl)-1H-imidazol-2-yl]-5-bromo-3-(pyrrolin-1-ylsulfonyl)-1H-indole;
methyl 5-{2-[5-bromo-3-(pyrrolidin-1-ylsulfonyl)-1H-indol-2-yl]-1H-imidazol-4-yl}-2-hydroxybenzoate;
5-bromo-2-[4-(4-nitrophenyl)-1H-imidazol-2-yl]-3-(pyrrolidin-1-ylsulfonyl)-1H-indole;
4-{2-[5-bromo-3-(pyrrolidin-1-ylsulfonyl)-1H-indol-2-yl]-1H-imidazol-4-yl}benzonitrile;
5-chloro-3-[(cyclobutylmethyl)sulfonyl]-2-(5-methyl-1H-imidazol-2-yl)-1H-indole;
5-chloro-3-[(cyclopentyl)sulfonyl]-2-(5-methyl-1H-imidazol-2-yl)-1H-indole;
5-chloro-3-[(2-methylbutyl)sulfonyl]-2-(5-methyl-1H-imidazol-2-yl)-1H-indole; and
5-chloro-3-[(pent-3-yl)sulfonyl]-2-(5-methyl-1H-imidazol-2-yl)-1H-indole.

15. A pharmaceutical composition comprising an effective amount of a compound according to claim 1, or a pharmaceutically acceptable salt thereof, and a pharmaceutically acceptable carrier.

16. A pharmaceutical combination which is (i) a compound according to claim 1, or a pharmaceutically acceptable salt thereof, and (ii) an HIV antiviral agent selected from the group consisting of HIV protease inhibitors, nucleoside HIV reverse transcriptase inhibitors, and HIV integrase inhibitors; wherein the compound of (i) or its pharmaceutically acceptable salt and the HIV antiviral agent of (ii) are each employed in an amount that renders the combination effective for the treatment of HIV infection or the treatment of AIDS.

17. A method for the treatment HIV infection, or the treatment of AIDS, wherein the method comprises administering to a subject in need thereof an effective amount of a compound of Formula I, or a pharmaceutically acceptable salt thereof, as defined in claim 1.

18. (canceled)

19. (canceled)
